(19) United States
(12) Patent Application Publication
(10) Pub. No.: US 2024/0260882 A1
(43) Pub. Date: Aug. 8, 2024

(54) ELECTROCARDIOGRAM ANALYSIS METHOD AND APPARATUS, ELECTRONIC DEVICE AND STORAGE MEDIUM

(71) Applicant: Hefei Heart Voice Health Technology Co., Ltd., Hefei (CN)

(72) Inventors: Shijia GENG, Hefei (CN); Shenda HONG, Hefei (CN); Guodong WEI, Hefei (CN); Kai WANG, Hefei (CN); Deyun ZHANG, Hefei (CN); Zhaoji FU, Hefei (CN); Rongbo ZHOU, Hefei (CN); Jie YU, Hefei (CN); Yanqi E, Hefei (CN); Xinyu QI, Hefei (CN)

(73) Assignee: Hefei Heart Voice Health Technology Co., Ltd., Hefei (CN)

(21) Appl. No.: 18/565,094

(22) PCT Filed: Jun. 13, 2022
(86) PCT No.: PCT/CN2022/098428

§ 371 Date: Nov. 29, 2023

(51) Int. Cl. A61B 5/346; A61B 5/00

(52) U.S. Cl. CPC A61B 5/346; A61B 5/7267; A61B 5/7275

(57) ABSTRACT

An electrocardiogram analysis method and apparatus, an electronic device and a storage medium are provided. In the method, at least one electrocardiogram data segment to be analyzed of a target user is input into a first electrocardiogram analysis model for analysis, so as to generate heart disease diagnosis result information of the target user; and optionally, when the heart disease diagnosis result information indicates that the probability of the target user suffering from a specific heart disease is relatively low, that is, when the electrocardiogram data segment to be analyzed is an electrocardiogram that looks relatively normal, whether the target user suffers in a paroxysmal manner from the heart disease is further determined. That is, the probability of the target user having the symptom corresponding to the heart disease in the future is predicted to provide early warning information for a future physical health condition of the target user.

### CROSS REFERENCE TO THE RELATED APPLICATIONS

This application is the national phase entry of International Application No. PCT/CN2022/098428, filed on Jun. 13, 2022, the entire contents of which are incorporated herein by reference.

### TECHNICAL FIELD

Embodiments of the present disclosure relate to the technical field of electrocardiogram analysis, and in particular, to an electrocardiogram analysis method and apparatus, an electronic device and a storage medium.

### BACKGROUND

The heart is one of the most important organs of the human body. In order to detect whether the heart function is normal, at present, an electrocardiogramanerally used to perform electrocardiogram examination on the heart function of the human body in a hospital so as to obtain electrocardiogram data, or a portable electrocardiogram acquisition device may be used to perform electrocardiogram examination on the heart function of the human body in a non-specific environment (for example, it is not necessarily limited to the hospital) so as to obtain electrocardiogram data.

### SUMMARY

Embodiments of the present disclosure relate to the technical field of electrocardiogram analysis, and specifically relate to an electrocardiogram analysis method and apparatus, an electronic device and a storage medium.

In a first aspect, embodiments of the present disclosure provide an electrocardiogram analysis method, including: acquiring at least one electrocardiogram data segment to be analyzed of a target user; inputting each electrocardiogram data segment to be analyzed into a pre-trained first electrocardiogram analysis model to obtain a heart disease suffering probability vector corresponding to the electrocardiogram data segment to be analyzed, wherein the heart disease suffering probability vector is used for characterizing a probability of suffering from each of K preset heart diseases, the first electrocardiogram analysis model is used for characterizing a correspondence between the electrocardiogram data segment and the heart disease suffering probability vector, and K is a positive integer; and generating heart disease diagnosis result information of the target user on the basis of the heart disease suffering probability vector corresponding to each electrocardiogram data segment to be analyzed.

In some alternative embodiments, the first electrocardiogram analysis model is pre-trained with the following first training steps: acquiring a first training data set, wherein first training data includes a sample electrocardiogram data segment and a corresponding labeled heart disease suffering probability vector, and the labeled heart disease suffering probability vector in the first training data is used for indicating a probability of a person, whom the sample electrocardiogram data segment in the first training data corresponds to and on whom collection is performed, suffering from each preset heart disease; training an initial first electrocardiogram analysis model on the basis of the first training data set; and determining the initial first electrocardiogram analysis model trained as the pre-trained first electrocardiogram analysis model.

In some alternative embodiments, the generating heart disease diagnosis result information of the target user on the basis of the heart disease suffering probability vector corresponding to each electrocardiogram data segment to be analyzed includes: for each preset heart disease, performing the following first diagnosis result information generating operations: determining a heart disease suffering probability of the target user suffering from the preset heart disease according to a component corresponding to the preset heart disease in the heart disease suffering probability vector corresponding to each electrocardiogram data segment to be analyzed; determining heart disease diagnosis result information corresponding to the heart disease suffering probability of the target user suffering from the preset heart disease according to a correspondence between a disease suffering probability range and heart disease diagnosis result information corresponding to the preset heart disease; and generating heart disease diagnosis result information of the target user suffering from the preset heart disease using the determined heart disease diagnosis result information.

In some alternative embodiments, the determining a heart disease suffering probability of the target user suffering from the preset heart disease according to a component corresponding to the preset heart disease in the heart disease suffering probability vector corresponding to each electrocardiogram data segment to be analyzed includes: determining a mean value of components corresponding to the preset heart disease in the heart disease suffering probability vector corresponding to each electrocardiogram data segment to be analyzed as the heart disease suffering probability of the target user suffering from the preset heart disease; or ranking components corresponding to the preset heart disease in the heart disease suffering probability vector corresponding to each electrocardiogram data segment to be analyzed in an order from low to high, and determining a component ranked in a preset quantile as the heart disease suffering probability of the target user suffering from the preset heart disease.

In some alternative embodiments, the determining a heart disease suffering probability of the target user suffering from the preset heart disease according to a component corresponding to the preset heart disease in the heart disease suffering probability vector corresponding to each electrocardiogram data segment to be analyzed includes: ranking components corresponding to the preset heart disease in the heart disease suffering probability vector corresponding to each electrocardiogram data segment to be analyzed in an order from low to high; in response to determining that current application scenario is a less false positive scenario, determining a minimum value of the components corresponding to the preset heart disease in the heart disease suffering probability vector corresponding to each electrocardiogram data segment to be analyzed or a component ranked in a preset lower probability quantile as the heart disease suffering probability of the target user suffering from the preset heart disease; and in response to determining that the current application scenario is a less false negative scenario, determining a maximum value of the components corresponding to the preset heart disease in the heart disease suffering probability vector corresponding to each electrocardiogram data segment to be analyzed or a component ranked in a preset higher probability quantile as the heart disease suffering probability of the target user suffering from the preset heart disease.

In some alternative embodiments, the correspondence between a disease suffering probability range and heart disease diagnosis result information corresponding to the preset heart disease includes at least one of: a first correspondence for characterizing that a first disease suffering probability range corresponds to first diagnosis result information indicating that the preset heart disease is not diagnosed, wherein the first disease suffering probability range is less than a disease suffering probability threshold corresponding to the preset heart disease; and a second correspondence for characterizing that a second disease suffering probability range corresponds to second diagnosis result information indicating that the preset heart disease is diagnosed, wherein the second disease suffering probability range is greater than or equal to a disease suffering probability threshold corresponding to the preset heart disease.

In some alternative embodiments, the disease suffering probability threshold corresponding to each preset heart disease is obtained by the following disease suffering probability threshold determination steps: acquiring a test data set, wherein test data includes a sample electrocardiogram data segment and a labeled heart disease suffering probability vector, and the labeled heart disease suffering probability vector in the test data is used for indicating a probability of a person, whom the sample electrocardiogram data segment in the test data corresponds to and on whom collection is performed, suffering from each preset heart disease; inputting sample electrocardiogram data segments in each of the test data into the first electrocardiogram analysis model to obtain a heart disease suffering probability vector test result corresponding to the test data; and for each preset heart disease, performing the following disease suffering probability threshold determination operations: acquiring a set of candidate disease suffering probability thresholds corresponding to the preset heart disease; for each candidate disease suffering probability threshold acquired, performing the following statistical operations: according to whether a vector component corresponding to the preset heart disease in the heart disease suffering probability vector test result corresponding to each of the test data is greater than the candidate disease suffering probability threshold, and whether a vector component corresponding to the preset heart disease in a labeled heart disease suffering probability vector in the corresponding test data is greater than the candidate disease suffering probability threshold, counting a sensitivity and specificity corresponding to the preset heart disease and the candidate disease suffering probability threshold; in response to determining that the current application scenario is a less false negative scenario, ranking the candidate disease suffering probability thresholds in the set of candidate disease suffering probability thresholds corresponding to the preset heart disease in an order of the corresponding sensitivity from high to low; determining a candidate disease suffering probability threshold ranked at a preset higher sensitivity ranking position in the set of candidate disease suffering probability thresholds corresponding to the preset heart disease as a heart disease suffering probability threshold corresponding to the preset heart disease; in response to determining that the current application scenario is a less false positive scenario, ranking the candidate disease suffering probability thresholds in the set of candidate disease suffering probability thresholds corresponding to the preset heart disease in an order of the corresponding specificity from high to low; and determining a candidate disease suffering probability threshold ranked at a preset higher specificity ranking position in the set of candidate disease suffering probability thresholds corresponding to the preset heart disease as a heart disease suffering probability threshold corresponding to the preset heart disease.

In some alternative embodiments, the generating heart disease diagnosis result information of the target user on the basis of the heart disease suffering probability vector corresponding to each electrocardiogram data segment to be analyzed includes: for each preset heart disease, performing the following second diagnosis result information generating operations: acquiring a set of disease suffering probability ranges corresponding to the preset heart disease; for each acquired disease suffering probability range, determining a proportion of data segments corresponding to the disease suffering probability range, wherein the proportion of data segments corresponding to the disease suffering probability range is a proportion of the number of components of the heart disease suffering probability vector belonging to the disease suffering probability range in the components corresponding to the preset heart disease in the heart disease suffering probability vector corresponding to each electrocardiogram data segment to be analyzed divided by the number of the electrocardiogram data segments to be analyzed; according to a correspondence between a disease suffering probability range and heart disease diagnosis result information corresponding to the preset heart disease, determining heart disease diagnosis result information corresponding to the disease suffering probability range with the largest proportion of the corresponding data segments; and generating heart disease diagnosis result information of the target user suffering from the preset heart disease using the determined heart disease diagnosis result information.

In some alternative embodiments, the generating heart disease diagnosis result information of the target user on the basis of the heart disease suffering probability vector corresponding to each electrocardiogram data segment to be analyzed includes: for each preset heart disease, in response to determining that a proportion of diagnosis electrocardiogram data segments corresponding to the preset heart disease is not less than a diagnosis proportion threshold corresponding to the preset heart disease, labeling the preset heart disease as a diagnosed heart disease, wherein the proportion of diagnosis electrocardiogram data segments corresponding to the preset heart disease is a proportion of the number of diagnosis electrocardiogram data segments corresponding to the preset heart disease divided by the total number of the electrocardiogram data segments to be analyzed, and the number of diagnosis electrocardiogram data segments corresponding to the preset heart disease is the number of components corresponding to the preset heart disease in a heart disease suffering probability vector corresponding to each electrocardiogram data segment to be analyzed, which are greater than a disease suffering probability threshold corresponding to the preset heart disease; and generating heart disease diagnosis result information indicating that the target user is diagnosed with a diagnosed heart disease among the preset heart diseases, which is labeled as a diagnosed heart disease.

In some alternative embodiments, the method further includes: for each of M preset paroxysmal heart diseases, in response to the target user's heart disease diagnosis result information indicating that a probability of the target user suffering from a heart disease corresponding to the preset paroxysmal heart disease falls within a preset lower disease suffering probability range, performing the following first paroxysmal heart disease prediction operations for the preset paroxysmal heart disease: calculating a probability vector distance between a heart disease suffering probability vector of the target user and a reference paroxysmal heart disease suffering probability vector corresponding to the preset paroxysmal heart disease; and generating paroxysmal heart disease diagnosis result information for indicating that the target user suffers from the preset paroxysmal disease in response to determining that the probability vector distance is less than a probability vector distance threshold corresponding to the preset paroxysmal heart disease, wherein M is a positive integer less than or equal to K, and heart diseases corresponding to the M preset paroxysmal heart diseases belong to the K preset heart diseases.

In some alternative embodiments, the first paroxysmal heart disease prediction operations further include: in response to determining that the probability vector distance is not less than the probability vector distance threshold corresponding to the preset paroxysmal heart disease, generating paroxysmal heart disease diagnosis result information indicating that the target user does not suffer from the preset paroxysmal disease.

In some alternative embodiments, the reference paroxysmal heart disease suffering probability vector corresponding to each preset paroxysmal heart disease is obtained by performing the following probability vector generation steps for each of the preset paroxysmal heart diseases: acquiring a set of unacknowledged condition electrocardiogram data segments corresponding to the preset paroxysmal heart disease, wherein each of the unacknowledged condition electrocardiogram data segments is an electrocardiogram data segment obtained after segmenting unacknowledged condition electrocardiogram data, and the unacknowledged condition electrocardiogram data is electrocardiogram data of electrocardiogram examination on a subject diagnosed with a heart disease corresponding to the paroxysmal heart disease, which is labeled as the subject corresponding to the unacknowledged condition electrocardiogram data does not suffer from the heart disease corresponding to the paroxysmal heart disease; inputting each unacknowledged condition electrocardiogram data segment into the first electrocardiogram analysis model to obtain a corresponding heart disease suffering probability vector; for each of the unacknowledged condition electrocardiogram data segments, determining a probability vector average distance corresponding to the unacknowledged condition electrocardiogram data segment, wherein the probability vector average distance corresponding to the unacknowledged condition electrocardiogram data segment is an average distance between a heart disease suffering probability vector corresponding to the unacknowledged condition electrocardiogram data segment and heart disease suffering probability vectors corresponding to other unacknowledged condition electrocardiogram data segments in the unacknowledged condition electrocardiogram data segment set except for the unacknowledged condition electrocardiogram data segment; determining a central unacknowledged condition electrocardiogram data segment in each of the unacknowledged condition electrocardiogram data segments on the basis of a probability vector average distance corresponding to each unacknowledged condition electrocardiogram data segment; and determining a heart disease suffering probability vector corresponding to the central unacknowledged condition electrocardiogram data segment as a reference paroxysmal heart disease suffering probability vector corresponding to the paroxysmal heart disease.

In some alternative embodiments, the probability vector distance threshold corresponding to each of the M preset paroxysmal heart diseases is obtained by: ranking the unacknowledged condition electrocardiogram data segments in an order of the corresponding probability vector average distance from high to low; determining an unacknowledged condition electrocardiogram data segment, ranked at a preset boundary probability vector average distance ranking position, of the unacknowledged condition electrocardiogram data segments as a boundary unacknowledged condition electrocardiogram data segment; and for each of the M preset paroxysmal heart diseases, determining a component, which is corresponding to the heart disease corresponding to the paroxysmal heart disease, of a heart disease suffering probability vector corresponding to the boundary unacknowledged condition electrocardiogram data segment as a probability vector distance threshold corresponding to the paroxysmal heart disease.

In some alternative embodiments, the method further includes: for each of M preset paroxysmal heart diseases, in response to the target user's heart disease diagnosis result information indicating that a probability of the target user suffering from a heart disease corresponding to the preset paroxysmal heart disease falls within a preset lower disease suffering probability range, performing the following second paroxysmal heart disease prediction operations: inputting each electrocardiogram data segment to be analyzed into a pre-trained second electrocardiogram analysis model corresponding to the preset paroxysmal heart disease to obtain a paroxysmal heart disease prediction result corresponding to the preset paroxysmal heart disease and the electrocardiogram data segment to be analyzed for characterizing whether the preset paroxysmal heart disease exists, wherein the second electrocardiogram analysis model corresponding to the preset paroxysmal heart disease is used for characterizing a correspondence between the electrocardiogram data segment and the paroxysmal heart disease prediction result; and generating a paroxysmal heart disease prediction result of the target user for the preset paroxysmal heart disease on the basis of the paroxysmal heart disease prediction result corresponding to the preset paroxysmal heart disease and each electrocardiogram data segment to be analyzed, wherein M is a positive integer less than or equal to K, and heart diseases corresponding to the M preset paroxysmal heart diseases belong to the K preset heart diseases. In some alternative embodiments, the generating a paroxysmal heart disease prediction result of the target user for the preset paroxysmal heart disease on the basis of the paroxysmal heart disease prediction result corresponding to the preset paroxysmal heart disease and each electrocardiogram data segment to be analyzed includes: determining whether a paroxysmal heart disease prediction result indicating suffering from the preset paroxysmal heart disease exists in the paroxysmal heart disease prediction results corresponding to the preset paroxysmal heart disease and each electrocardiogram data segment to be analyzed; and in response to determining existence, generating a paroxysmal heart disease prediction result indicating that the target user suffers from the preset paroxysmal heart disease.

In some alternative embodiments, the generating a paroxysmal heart disease prediction result of the target user for the preset paroxysmal heart disease on the basis of the paroxysmal heart disease prediction result corresponding to the preset paroxysmal heart disease and each electrocardiogram data segment to be analyzed further includes: in response to determining absence, generating a paroxysmal heart disease prediction result indicating that the target user does not suffer from the preset paroxysmal heart disease.

In some alternative embodiments, the generating a paroxysmal heart disease prediction result of the target user for the preset paroxysmal heart disease on the basis of the paroxysmal heart disease prediction result corresponding to the preset paroxysmal heart disease and each electrocardiogram data segment to be analyzed includes: generating a paroxysmal heart disease prediction result indicating that the target user suffers from the preset paroxysmal heart disease in response to determining that a proportion of diagnosis prediction results corresponding to the preset paroxysmal heart disease is greater than a diagnosis prediction result proportion threshold corresponding to the preset paroxysmal heart disease, wherein the proportion of diagnosis prediction results corresponding to the preset paroxysmal heart disease is a proportion of the number of diagnosis prediction results corresponding to the preset paroxysmal heart disease divided by the total number of the electrocardiogram data segments to be analyzed, and the number of the diagnosis prediction results corresponding to the preset paroxysmal heart disease is the number of paroxysmal heart disease prediction results indicating suffering from the preset paroxysmal heart disease in the paroxysmal heart disease prediction results corresponding to the preset paroxysmal heart disease and the electrocardiogram data segments to be analyzed.

In some alternative embodiments, the generating a paroxysmal heart disease prediction result of the target user for the preset paroxysmal heart disease on the basis of the paroxysmal heart disease prediction result corresponding to the preset paroxysmal heart disease and each electrocardiogram data segment to be analyzed further includes: generating a paroxysmal heart disease prediction result indicating that the target user does not suffer from the preset paroxysmal heart disease in response to determining that the proportion of diagnosis prediction results corresponding to the preset paroxysmal heart disease is not greater than the diagnosis prediction result proportion threshold corresponding to the preset paroxysmal heart disease. In some alternative embodiments, the acquiring at least one electrocardiogram data segment to be analyzed of a target user includes: acquiring electrocardiogram data to be analyzed of a target user; and segmenting the electrocardiogram data to be analyzed to obtain at least one electrocardiogram data segment to be analyzed.

In some alternative embodiments, before the segmenting the electrocardiogram data to be analyzed to obtain at least one electrocardiogram data segment to be analyzed, the method further includes:
resampling the electrocardiogram data to be analyzed so that a sampling frequency of the electrocardiogram data to be analyzed is a preset sampling frequency.

In some alternative embodiments, the segmenting the electrocardiogram data to be analyzed to obtain at least one electrocardiogram data segment to be analyzed includes: performing average segmentation processing on the electrocardiogram data to be analyzed to obtain at least one electrocardiogram data segment to be analyzed, wherein each electrocardiogram data segment to be analyzed includes electrocardiogram data of F frames, and F is a positive integer.

In some alternative embodiments, the K preset heart diseases are K heart diseases selected from a preset heart disease set including: sinus tachycardia, sinus bradycardia, premature atrial contraction, premature junctional contraction, premature ventricular contraction, supraventricular tachycardia, ventricular tachycardia, atrial flutter, atrial fibrillation, atrial escape, junctional escape, ventricular escape, right bundle branch block, sinus arrhythmia, sinus arrest, supraventricular premature beats, paired supraventricular premature beats, bigeminy coupled rhythm of supraventricular premature beats, trigeminy of supraventricular premature beats, ventricular premature beats, paired ventricular premature beats, bigeminy coupled rhythm of ventricular premature beats, trigeminy of ventricular premature beats, supraventricular escape beats, pre-excitation syndrome, ventricular flutter, ventricular fibrillation, ventricular escape, first degree atrio-ventricular block, secondary degree atrio-ventricular block, third degree atrio-ventricular block, intra-ventricular block, left bundle branch block, complete right bundle branch block, conduction block in left forearm, left ventricular hypertrophy, right ventricular hypertrophy, left atrial hypertrophy and right atrial hypertrophy.

In some alternative embodiments, the M preset paroxysmal heart diseases are M paroxysmal heart diseases selected from a preset paroxysmal heart disease set including: paroxysmal sinus tachycardia, paroxysmal sinus bradycardia, paroxysmal premature atrial contraction, paroxysmal premature junctional contraction, paroxysmal premature ventricular contraction, paroxysmal supraventricular tachycardia, paroxysmal ventricular tachycardia, paroxysmal atrial flutter, paroxysmal atrial fibrillation, paroxysmal atrial escape, paroxysmal junctional escape, paroxysmal ventricular escape, paroxysmal sinus arrhythmia, paroxysmal sinus arrest and paroxysmal supraventricular premature beats.

In a second aspect, embodiments of the present disclosure provide an electrocardiogram analysis apparatus, including: a data acquisition unit, configured to acquire at least one electrocardiogram data segment to be analyzed of a target user; a data analysis unit, configured to input each electrocardiogram data segment to be analyzed into a pre-trained first electrocardiogram analysis model to obtain a heart disease suffering probability vector corresponding to the electrocardiogram data segment to be analyzed, wherein the heart disease suffering probability vector is used for characterizing a probability of suffering from each of K preset heart diseases, the first electrocardiogram analysis model is used for characterizing a correspondence between the electrocardiogram data segment and the heart disease suffering probability vector, and K is a positive integer; and a heart disease diagnosis result generation unit, configured to generate heart disease diagnosis result information of the target user on the basis of the heart disease suffering probability vector corresponding to each electrocardiogram data segment to be analyzed.

In some alternative embodiments, the first electrocardiogram analysis model is pre-trained with the following first training steps: acquiring a first training data set, wherein first training data includes a sample electrocardiogram data segment and a corresponding labeled heart disease suffering probability vector, and the labeled heart disease suffering probability vector in the first training data is used for indicating a probability of a person, whom the sample electrocardiogram data segment in the first training data corresponds to and on whom collection is performed, suffering from each preset heart disease; training an initial first electrocardiogram analysis model on the basis of the first training data set; and determining the initial first electrocardiogram analysis model trained as the pre-trained first electrocardiogram analysis model.

In some alternative embodiments, the heart disease diagnosis result generation unit is further configured to: for each preset heart disease, perform the following first diagnosis result information generating operations: determining a heart disease suffering probability of the target user suffering from the preset heart disease according to a component corresponding to the preset heart disease in the heart disease suffering probability vector corresponding to each electrocardiogram data segment to be analyzed; determining heart disease diagnosis result information corresponding to the heart disease suffering probability of the target user suffering from the preset heart disease according to a correspondence between a disease suffering probability range and heart disease diagnosis result information corresponding to the preset heart disease; and generating heart disease diagnosis result information of the target user suffering from the preset heart disease using the determined heart disease diagnosis result information. In some alternative embodiments, the determining a heart disease suffering probability of the target user suffering from the preset heart disease according to a component corresponding to the preset heart disease in the heart disease suffering probability vector corresponding to each electrocardiogram data segment to be analyzed includes: determining a mean value of components corresponding to the preset heart disease in the heart disease suffering probability vector corresponding to each electrocardiogram data segment to be analyzed as the heart disease suffering probability of the target user suffering from the preset heart disease; or ranking components corresponding to the preset heart disease in the heart disease suffering probability vector corresponding to each electrocardiogram data segment to be analyzed in an order from low to high, and determining a component ranked in a preset quantile as the heart disease suffering probability of the target user suffering from the preset heart disease.

In some alternative embodiments, the determining a heart disease suffering probability of the target user suffering from the preset heart disease according to a component corresponding to the preset heart disease in the heart disease suffering probability vector corresponding to each electrocardiogram data segment to be analyzed includes: ranking components corresponding to the preset heart disease in the heart disease suffering probability vector corresponding to each electrocardiogram data segment to be analyzed in an order from low to high; in response to determining that current application scenario is a less false positive scenario, determining a minimum value of the components corresponding to the preset heart disease in the heart disease suffering probability vector corresponding to each electrocardiogram data segment to be analyzed or a component ranked in a preset lower probability quantile as the heart disease suffering probability of the target user suffering from the preset heart disease; and in response to determining that the current application scenario is a less false negative scenario, determining a maximum value of the components corresponding to the preset heart disease in the heart disease suffering probability vector corresponding to each electrocardiogram data segment to be analyzed or a component ranked in a preset higher probability quantile as the heart disease suffering probability of the target user suffering from the preset heart disease.

In some alternative embodiments, the correspondence between a disease suffering probability range and heart disease diagnosis result information corresponding to the preset heart disease includes at least one of: a first correspondence for characterizing that a first disease suffering probability range corresponds to first diagnosis result information indicating that the preset heart disease is not diagnosed, wherein the first disease suffering probability range is less than a disease suffering probability threshold corresponding to the preset heart disease; and a second correspondence for characterizing that a second disease suffering probability range corresponds to second diagnosis result information indicating that the preset heart disease is diagnosed, wherein the second disease suffering probability range is greater than or equal to a disease suffering probability threshold corresponding to the preset heart disease.

In some alternative embodiments, the disease suffering probability threshold corresponding to each preset heart disease is obtained by the following disease suffering probability threshold determination steps: acquiring a test data set, wherein test data includes a sample electrocardiogram data segment and a labeled heart disease suffering probability vector, and the labeled heart disease suffering probability vector in the test data is used for indicating a probability of a person, whom the sample electrocardiogram data segment in the test data corresponds to and on whom collection is performed, suffering from each preset heart disease; inputting sample electrocardiogram data segments in each of the test data into the first electrocardiogram analysis model to obtain a heart disease suffering probability vector test result corresponding to the test data; and for each preset heart disease, performing the following disease suffering probability threshold determination operations: acquiring a set of candidate disease suffering probability thresholds corresponding to the preset heart disease; for each candidate disease suffering probability threshold acquired, performing the following statistical operations: according to whether a vector component corresponding to the preset heart disease in the heart disease suffering probability vector test result corresponding to each of the test data is greater than the candidate disease suffering probability threshold, and whether a vector component corresponding to the preset heart disease in a labeled heart disease suffering probability vector in the corresponding test data is greater than the candidate disease suffering probability threshold, counting a sensitivity and specificity corresponding to the preset heart disease and the candidate disease suffering probability threshold; in response to determining that the current application scenario is a less false negative scenario, ranking the candidate disease suffering probability thresholds in the set of candidate disease suffering probability thresholds corresponding to the preset heart disease in an order of the corresponding sensitivity from high to low; determining a candidate disease suffering probability threshold ranked at a preset higher sensitivity ranking position in the set of candidate disease suffering probability thresholds corresponding to the preset heart disease as a heart disease suffering probability threshold corresponding to the preset heart disease; in response to determining that the current application scenario is a less false positive scenario, ranking the candidate disease suffering probability thresholds in the set of candidate disease suffering probability thresholds corresponding to the preset heart disease in an order of the corresponding specificity from high to low; and determining a candidate disease suffering probability threshold ranked at a preset higher specificity ranking position in the set of candidate disease suffering probability thresholds corresponding to the preset heart disease as a heart disease suffering probability threshold corresponding to the preset heart disease.

In some alternative embodiments, the heart disease diagnosis result generation unit is further configured to: for each preset heart disease, perform the following second diagnosis result information generating operations: acquiring a set of disease suffering probability range corresponding to the preset heart disease; for each acquired disease suffering probability range, determining a proportion of data segments corresponding to the disease suffering probability range, wherein the proportion of data segments corresponding to the disease suffering probability range is a proportion of the number of components of the heart disease suffering probability vector belonging to the disease suffering probability range in the components corresponding to the preset heart disease in the heart disease suffering probability vector corresponding to each electrocardiogram data segment to be analyzed divided by the number of the electrocardiogram data segments to be analyzed; according to a correspondence between a disease suffering probability range and heart disease diagnosis result information corresponding to the preset heart disease, determining heart disease diagnosis result information corresponding to the disease suffering probability range with the largest proportion of the corresponding data segments; and generating heart disease diagnosis result information of the target user suffering from the preset heart disease using the determined heart disease diagnosis result information.

In some alternative embodiments, the heart disease diagnosis result generation unit is further configured to: for each preset heart disease, in response to determining that a proportion of diagnosis electrocardiogram data segments corresponding to the preset heart disease is not less than a diagnosis proportion threshold corresponding to the preset heart disease, label the preset heart disease as a diagnosed heart disease, wherein the proportion of diagnosis electrocardiogram data segments corresponding to the preset heart disease is a proportion of the number of diagnosis electrocardiogram data segments corresponding to the preset heart disease divided by the total number of the electrocardiogram data segments to be analyzed, and the number of diagnosis electrocardiogram data segments corresponding to the preset heart disease is the number of components corresponding to the preset heart disease in a heart disease suffering probability vector corresponding to each electrocardiogram data segment to be analyzed, which are greater than a disease suffering probability threshold corresponding to the preset heart disease; and generate heart disease diagnosis result information indicating that the target user is diagnosed with a diagnosed heart disease among the preset heart diseases, which is labeled as a diagnosed heart disease.

In some alternative embodiments, the apparatus further includes a first paroxysmal heart disease prediction unit, configured to: for each of M preset paroxysmal heart diseases, in response to the target user's heart disease diagnosis result information indicating that a probability of the target user suffering from a heart disease corresponding to the preset paroxysmal heart disease falls within a preset lower disease suffering probability range, perform the following first paroxysmal heart disease prediction operations for the preset paroxysmal heart disease: calculating a probability vector distance between a heart disease suffering probability vector of the target user and a reference paroxysmal heart disease suffering probability vector corresponding to the preset paroxysmal heart disease; and generating paroxysmal heart disease diagnosis result information for indicating that the target user suffers from the preset paroxysmal disease in response to determining that the probability vector distance is less than a probability vector distance threshold corresponding to the preset paroxysmal heart disease, wherein M is a positive integer less than or equal to K, and heart diseases corresponding to the M preset paroxysmal heart diseases belong to the K preset heart diseases.

In some alternative embodiments, the first paroxysmal heart disease prediction operations further include: in response to determining that the probability vector distance is not less than the probability vector distance threshold corresponding to the preset paroxysmal heart disease, generating paroxysmal heart disease diagnosis result information indicating that the target user does not suffer from the preset paroxysmal disease.

In some alternative embodiments, the reference paroxysmal heart disease suffering probability vector corresponding to each preset paroxysmal heart disease is obtained by performing the following probability vector generation steps for each of the preset paroxysmal heart diseases: acquiring a set of unacknowledged condition electrocardiogram data segments corresponding to the preset paroxysmal heart disease, wherein each of the unacknowledged condition electrocardiogram data segments is an electrocardiogram data segment obtained after segmenting unacknowledged condition electrocardiogram data, and the unacknowledged condition electrocardiogram data is electrocardiogram data of electrocardiogram examination on a subject diagnosed with a heart disease corresponding to the paroxysmal heart disease, which is labeled as the subject corresponding to the unacknowledged condition electrocardiogram data does not suffer from the heart disease corresponding to the paroxysmal heart disease; inputting each unacknowledged condition electrocardiogram data segment into the first electrocardiogram analysis model to obtain a corresponding heart disease suffering probability vector; for each of the unacknowledged condition electrocardiogram data segments, determining a probability vector average distance corresponding to the unacknowledged condition electrocardiogram data segment, wherein the probability vector average distance corresponding to the unacknowledged condition electrocardiogram data segment is an average distance between a heart disease suffering probability vector corresponding to the unacknowledged condition electrocardiogram data segment and heart disease suffering probability vectors corresponding to other unacknowledged condition electrocardiogram data segments in the unacknowledged condition electrocardiogram data segment set except for the unacknowledged condition electrocardiogram data segment; determining a central unacknowledged condition electrocardiogram data segment in each of the unacknowledged condition electrocardiogram data segments on the basis of a probability vector average distance corresponding to each unacknowledged condition electrocardiogram data segment; and determining a heart disease suffering probability vector corresponding to the central unacknowledged condition electrocardiogram data segment as a reference paroxysmal heart disease suffering probability vector corresponding to the paroxysmal heart disease.

In some alternative embodiments, the probability vector distance threshold corresponding to each of the M preset paroxysmal heart diseases is obtained by: ranking the unacknowledged condition electrocardiogram data segments in an order of the corresponding probability vector average distance from high to low; determining an unacknowledged condition electrocardiogram data segment, ranked at a preset boundary probability vector average distance ranking position, of the unacknowledged condition electrocardiogram data segments as a boundary unacknowledged condition electrocardiogram data segment; and for each of the M preset paroxysmal heart diseases, determining a component, which is corresponding to the heart disease corresponding to the paroxysmal heart disease, of a heart disease suffering probability vector corresponding to the boundary unacknowledged condition electrocardiogram data segment as a probability vector distance threshold corresponding to the paroxysmal heart disease.

In some alternative embodiments, the apparatus further includes: a second paroxysmal heart disease prediction unit, configured to: for each of M preset paroxysmal heart diseases, in response to the target user's heart disease diagnosis result information indicating that a probability of the target user suffering from a heart disease corresponding to the preset paroxysmal heart disease falls within a preset lower disease suffering probability range, perform the following second paroxysmal heart disease prediction operations: inputting each electrocardiogram data segment to be analyzed into a pre-trained second electrocardiogram analysis model corresponding to the preset paroxysmal heart disease to obtain a paroxysmal heart disease prediction result corresponding to the preset paroxysmal heart disease and the electrocardiogram data segment to be analyzed for characterizing whether the preset paroxysmal heart disease exists, wherein the second electrocardiogram analysis model corresponding to the preset paroxysmal heart disease is used for characterizing a correspondence between the electrocardiogram data segment and the paroxysmal heart disease prediction result; and generating a paroxysmal heart disease prediction result of the target user for the preset paroxysmal heart disease on the basis of the paroxysmal heart disease prediction result corresponding to the preset paroxysmal heart disease and each electrocardiogram data segment to be analyzed, wherein M is a positive integer less than or equal to K, and heart diseases corresponding to the M preset paroxysmal heart diseases belong to the K preset heart diseases.

In some alternative embodiments, the generating a paroxysmal heart disease prediction result of the target user for the preset paroxysmal heart disease on the basis of the paroxysmal heart disease prediction result corresponding to the preset paroxysmal heart disease and each electrocardiogram data segment to be analyzed includes: determining whether a paroxysmal heart disease prediction result indicating suffering from the preset paroxysmal heart disease exists in the paroxysmal heart disease prediction results corresponding to the preset paroxysmal heart disease and each electrocardiogram data segment to be analyzed; and in response to determining existence, generating a paroxysmal heart disease prediction result indicating that the target user suffers from the preset paroxysmal heart disease.

In some alternative embodiments, the generating a paroxysmal heart disease prediction result of the target user for the preset paroxysmal heart disease on the basis of the paroxysmal heart disease prediction result corresponding to the preset paroxysmal heart disease and each electrocardiogram data segment to be analyzed further includes: in response to determining absence, generating a paroxysmal heart disease prediction result indicating that the target user does not suffer from the preset paroxysmal heart disease.

In some alternative embodiments, the generating a paroxysmal heart disease prediction result of the target user for the preset paroxysmal heart disease on the basis of the paroxysmal heart disease prediction result corresponding to the preset paroxysmal heart disease and each electrocardiogram data segment to be analyzed includes: generating a paroxysmal heart disease prediction result indicating that the target user suffers from the preset paroxysmal heart disease in response to determining that a proportion of diagnosis prediction results corresponding to the preset paroxysmal heart disease is greater than a diagnosis prediction result proportion threshold corresponding to the preset paroxysmal heart disease, wherein the proportion of diagnosis prediction results corresponding to the preset paroxysmal heart disease is a proportion of the number of diagnosis prediction results corresponding to the preset paroxysmal heart disease divided by the total number of the electrocardiogram data segments to be analyzed, and the number of the diagnosis prediction results corresponding to the preset paroxysmal heart disease is the number of paroxysmal heart disease prediction results indicating suffering from the preset paroxysmal heart disease in the paroxysmal heart disease prediction results corresponding to the preset paroxysmal heart disease and the electrocardiogram data segments to be analyzed.

In some alternative embodiments, the generating a paroxysmal heart disease prediction result of the target user for the preset paroxysmal heart disease on the basis of the paroxysmal heart disease prediction result corresponding to the preset paroxysmal heart disease and each electrocardiogram data segment to be analyzed further includes: generating a paroxysmal heart disease prediction result indicating that the target user does not suffer from the preset paroxysmal heart disease in response to determining that the proportion of diagnosis prediction results corresponding to the preset paroxysmal heart disease is not greater than the diagnosis prediction result proportion threshold corresponding to the preset paroxysmal heart disease. In some alternative embodiments, the data acquisition unit is further configured to: acquire electrocardiogram data to be analyzed of a target user; and segment the electrocardiogram data to be analyzed to obtain at least one electrocardiogram data segment to be analyzed.

In some alternative embodiments, the data acquisition unit is further configured to: before the segmenting the electrocardiogram data to be analyzed to obtain at least one electrocardiogram data segment to be analyzed, resample the electrocardiogram data to be analyzed so that a sampling frequency of the electrocardiogram data to be analyzed is a preset sampling frequency. In some alternative embodiments, the segmenting the electrocardiogram data to be analyzed to obtain at least one electrocardiogram data segment to be analyzed includes: performing average segmentation processing on the electrocardiogram data to be analyzed to obtain at least one electrocardiogram data segment to be analyzed, wherein each electrocardiogram data segment to be analyzed includes electrocardiogram data of F frames, and F is a positive integer.

In some alternative embodiments, the K preset heart diseases are K heart diseases selected from a preset heart disease set including: sinus tachycardia, sinus bradycardia, premature atrial contraction, premature junctional contraction, premature ventricular contraction, supraventricular tachycardia, ventricular tachycardia, atrial flutter, atrial fibrillation, atrial escape, junctional escape, ventricular escape, right bundle branch block, sinus arrhythmia, sinus arrest, supraventricular premature beats, paired supraventricular premature beats, bigeminy coupled rhythm of supraventricular premature beats, trigeminy of supraventricular premature beats, ventricular premature beats, paired ventricular premature beats, bigeminy coupled rhythm of ventricular premature beats, trigeminy of ventricular premature beats, supraventricular escape beats, pre-excitation syndrome, ventricular flutter, ventricular fibrillation, ventricular escape, first degree atrio-ventricular block, secondary degree atrio-ventricular block, third degree atrio-ventricular block, intra-ventricular block, left bundle branch block, complete right bundle branch block, conduction block in left forearm, left ventricular hypertrophy, right ventricular hypertrophy, left atrial hypertrophy and right atrial hypertrophy.

In some alternative embodiments, the M preset paroxysmal heart diseases are M paroxysmal heart diseases selected from a preset paroxysmal heart disease set including: paroxysmal sinus tachycardia, paroxysmal sinus bradycardia, paroxysmal premature atrial contraction, paroxysmal premature junctional contraction, paroxysmal premature ventricular contraction, paroxysmal supraventricular tachycardia, paroxysmal ventricular tachycardia, paroxysmal atrial flutter, paroxysmal atrial fibrillation, paroxysmal atrial escape, paroxysmal junctional escape, paroxysmal ventricular escape, paroxysmal sinus arrhythmia, paroxysmal sinus arrest and paroxysmal supraventricular premature beats.

In a third aspect, embodiments of the present disclosure provide an electronic device, including: one or more processors; and a storage apparatus having one or more programs stored thereon, wherein the one or more programs, when executed by the one or more processors, cause the one or more processors to perform the method as described in any one of the implementations in the first aspect.

In a fourth aspect, embodiments of the present disclosure provide a computer-readable storage medium, having a computer program stored thereon, wherein the computer program, when executed by one or more processors, implements the method as described in any one of the implementations in the first aspect.

According to the electrocardiogram analysis method and apparatus, the electronic device and the storage medium provided by the embodiments of the present disclosure, at least one electrocardiogram data segment to be analyzed of the target user is first acquired. Then, each electrocardiogram data segment to be analyzed is input into the pre-trained first electrocardiogram analysis model to obtain the heart disease suffering probability vector corresponding to each electrocardiogram data segment to be analyzed. Finally, on the basis of the heart disease suffering probability vector corresponding to each electrocardiogram data segment to be analyzed, the heart disease diagnosis result information of the target user is generated. Thereby heart disease diagnosis result information of the target user is obtained by analyzing at least one piece of electrocardiogram data of the target user.

### BRIEF DESCRIPTION OF THE DRAWINGS

Other features, objects, and advantages of the present disclosure will become more apparent upon reading the detailed description of non-limiting embodiments made with reference to the following drawings. The drawings are only for purposes of illustrating specific embodiments and are not to be construed as limiting the present disclosure. In the drawings:

FIG. 1 is an exemplary system architecture diagram to which one embodiment of the present disclosure may be applied;

FIG. 2A is a flowchart of one embodiment of an electrocardiogram analysis method according to the present disclosure;

FIG. 2B is an exploded flowchart of one embodiment of step 203 according to the present disclosure;

FIG. 2C is an exploded flowchart of another embodiment of step 203 according to the present disclosure;

FIG. 2D is an exploded flowchart of yet another embodiment of step 203 according to the present disclosure;

FIG. 3 is a flowchart of one embodiment of a first training step according to the present disclosure;

FIG. 4 is a flowchart of one embodiment of a disease suffering probability threshold determination step according to the present disclosure;

FIG. 5 is a flowchart of another embodiment of the electrocardiogram analysis method according to the present disclosure;

FIG. 6 is a flowchart of one embodiment of a probability vector generation step according to the present disclosure;

FIG. 7 is a flowchart of one embodiment of a probability vector distance threshold determination step according to the present disclosure;

FIG. 8A is a flowchart of another embodiment of the electrocardiogram analysis method according to the present disclosure;

FIG. 8B is an exploded flowchart of one embodiment of step 8042 according to the present disclosure;

FIG. 8C is an exploded flowchart of yet another embodiment of step 8042 according to the present disclosure;

FIG. 9 is a flowchart of one embodiment of a second training step according to the present disclosure;

FIG. 10 is a schematic structural diagram of one embodiment of an electrocardiogram analysis apparatus according to the present disclosure; and

FIG. 11 is a schematic structural diagram of a computer system of one embodiment of an electronic device according to the present disclosure.

### DETAILED DESCRIPTION OF THE EMBODIMETNS

The present disclosure will be described below in further detail with reference to the accompanying drawings and embodiments. It should be understood that the specific embodiments described herein are illustrative only and are not restrictive of the present disclosure. It is further noted that, for ease of description, only parts that are relevant to the present disclosure are shown in the drawings.

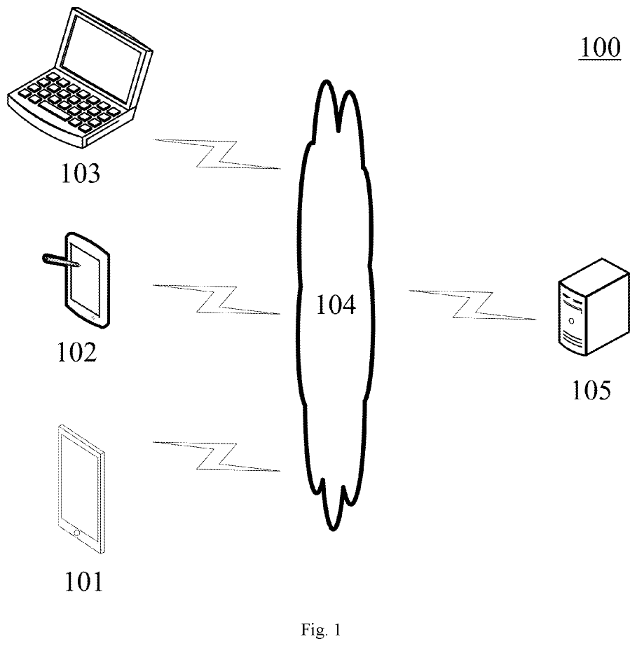

It should be noted that embodiments in the present disclosure and features in the embodiments may be combined with each other without conflict. The present disclosure will be described below in detail in connection with embodiments with reference to the accompanying drawings. FIG. 1 illustrates an exemplary system architecture 100 to which an embodiment of an electrocardiogram analysis method or an electrocardiogram analysis apparatus of the present disclosure may be applied.

As shown in FIG. 1, the system architecture 100 may include clients 101, 102, and 103, a network 104, and a server 105. The network 104 serves as a medium for providing communication links between the clients 101, 102, and 103 and the server 105. The network 104 may include various connection types, such as wired, wireless communication links, or fiber optic cables, among others.

A user may interact with the server 105 through the network 104 using the clients 101, 102, and 103 to receive or send messages, etc. Various communication client applications can be installed on the clients 101, 102, and 103, such as an electrocardiogram acquisition application, an electrocardiogram analysis application, a remote inquiry application, a medical information consultation application, a health condition monitoring application, a web browser application, a shopping application, a search application, an instant communication tool, a mailbox client, social platform software, etc.

The clients 101, 102, and 103 may be hardware or software. When the clients 101, 102, and 103 are hardware, the clients 101, 102, and 103 may be a variety of electronic devices with a display screen, including but not limited to smart phones, tablet computers, e-book readers, MP3 players (Moving Picture Experts Group Audio Layer III), MP4 (Moving Picture Experts Group Audio Layer IV) players, laptops and desktop computers, etc. When the clients 101, 102, and 103 are software, the clients 101, 102, and 103 can be installed in the electronic devices listed above. They may be implemented as multiple software or software modules (e.g. to provide portable electrocardiogram acquisition and analysis services) or as a single software or software module. No specific limitations are made herein.

In some cases, an electrocardiogram analysis method provided by the present disclosure may be performed by the clients 101, 102, and 103, and accordingly, the electrocardiogram analysis apparatus may be arranged in the clients 101, 102, and 103. In this case, the system architecture 100 may not include the server 105.

In some cases, the electrocardiogram analysis method provided by the present disclosure can be jointly executed by the clients 101, 102 and 103 and the server 105, for example, the step of “acquiring at least one electrocardiogram data segment to be analyzed of a target user” can be executed by the clients 101, 102 and 103, and the step of “inputting each electrocardiogram data segment to be analyzed into a pre-trained first electrocardiogram analysis model to obtain a heart disease suffering probability vector corresponding to the electrocardiogram data segment to be analyzed” can be executed by the server 105, which is not limited in the present disclosure. Accordingly, the electrocardiogram analysis apparatus may also be provided in the clients 101, 102, and 103 and the server 105, respectively.

In some cases, the electrocardiogram analysis method provided by the present disclosure may be performed by the server 105, and accordingly, the electrocardiogram analysis apparatus may also be arranged in the server 105, and in this case, the system architecture 100 may not include the clients 101, 102, and 103.

The server 105 may be a server providing various services, such as a background server providing support for electrocardiogram analysis type applications displayed on the clients 101, 102, and 103 or web pages providing electrocardiogram analysis type services. The background server may perform processing such as analysis on the received data such as an electrocardiogram analysis request, and feed back the processing result (such as heart disease diagnosis result information) to the clients.

It should be noted that the server 105 may be hardware or software. When the server 105 is hardware, it may be implemented as a distributed server cluster of multiple servers or as a single server. When the server 105 is software, it may be implemented as multiple software or software modules (e.g. to provide distributed services) or as a single software or software module. No specific limitations are made herein.

It should be understood that the number of clients, networks, and servers in FIG. 1 is merely illustrative. There may be any number of clients, networks, and servers as desired for implementation.

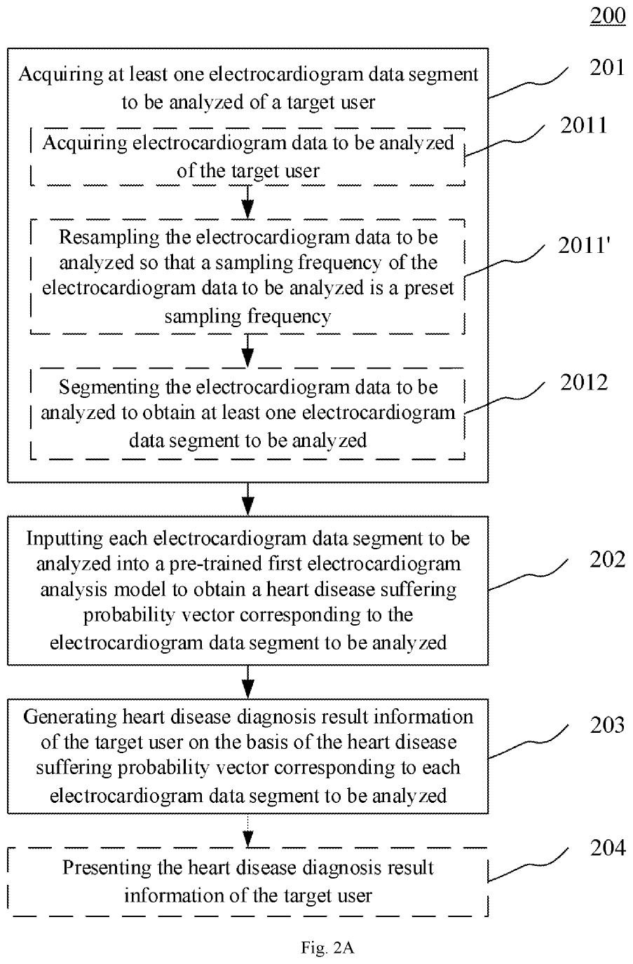

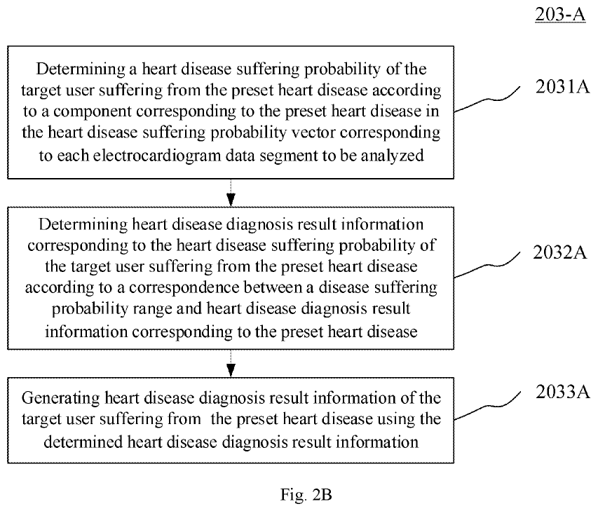

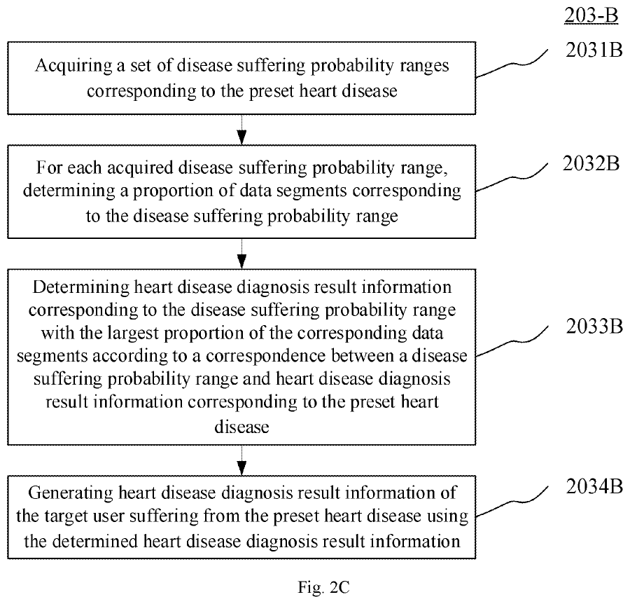

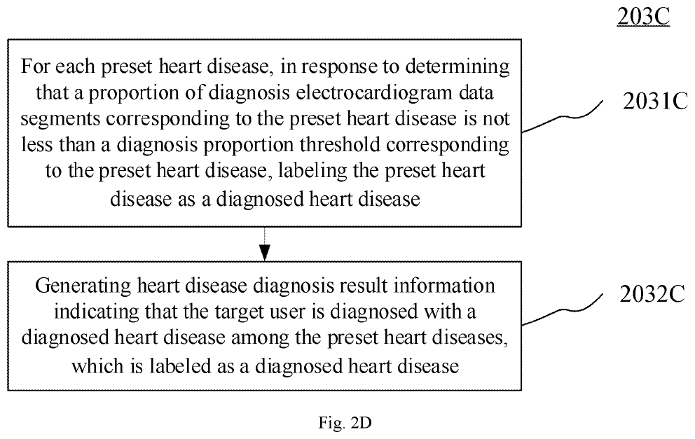

With continued reference to FIG. 2A, a flow 200 of one embodiment of an electrocardiogram analysis method according to the present disclosure is shown, the electrocardiogram analysis method including the following steps:

Step 201, at least one electrocardiogram data segment to be analyzed of a target user is acquired. In this embodiment, an executive subject of the electrocardiogram analysis method (e.g. the clients shown in FIG. 1) may acquire at least one electrocardiogram data segment to be analyzed of the target user locally or from other electronic devices connected to the executive subject through a network (e.g. a portable electrocardiogram acquisition device wirelessly connected to a client via Bluetooth).

Here, the target user may first undergo an electrocardiogram examination using an electrocardiogram a portable electrocardiogram acquisition device to obtain electrocardiogram data of the target user. The electrocardiogram data of the target user may include an electrocardiogram signal sequence of at least one lead. The at least one electrocardiogram data segment to be analyzed of the target user may be obtained after preprocessing on the basis of the electrocardiogram data of the target user.

In some alternative implementations, an L-lead electrocardiogram signal sequence may be included in each electrocardiogram data segment to be analyzed of the target user. Here, L may be a positive integer greater than or equal to 1. It will be appreciated that when L is 1, each electrocardiogram data segment to be analyzed of the target user is a single-lead electrocardiogram signal sequence. For most portable electrocardiogram acquisition devices, single-lead electrocardiogram signals are usually acquired. Therefore, electrocardiogram analysis can be performed on the target user's single-lead electrocardiogram signal sequence collected by the portable electrocardiogram acquisition device, and the corresponding heart disease diagnosis result information can be obtained. Since the portable electrocardiogram acquisition device is relatively portable, electrocardiogram signal acquisition and analysis can be performed at anytime and anywhere.

In some alternative implementations, step 201 may include the following steps 2011 and 2012: Step 2011, electrocardiogram data to be analyzed of the target user is acquired.

Optionally, the target user may perform electrocardiogram examination by himself/herself by using the portable electrocardiogram acquisition device, and then the executive subject may connect (e.g. via a Bluetooth wireless connection) the portable electrocardiogram acquisition device to acquire electrocardiogram data to be analyzed of the target user acquired by the portable electrocardiogram acquisition device. In this way, the acquired electrocardiogram data of the target user can be acquired and analyzed in time so as to generate the heart disease diagnosis result information, so that the heart disease diagnosis result information can be obtained after electrocardiogram examination is performed on the user at any time and anywhere, and timely analysis is performed, and then whether to go to the hospital to see a doctor or not can be determined according to the heart disease diagnosis result information.

Optionally, an electrocardiograman electrocardiogramn a hospital) can be used to perform an electrocardiogram examination on the target user to obtain electrocardiogram data to be analyzed of the target user, and a control host connected to the electrocardiogram be used to obtain the electrocardiogram data to be analyzed of the target user. In addition, the above-mentioned executive subject may also be connected to the control host connected to the electrocardiogramired or wireless network so as to acquire electrocardiogram data to be analyzed of the target user collected by the electrocardiogra

Step 2012, the electrocardiogram data to be analyzed is segmented to obtain at least one electrocardiogram data segment to be analyzed.

In order to make the electrocardiogram data segment to be analyzed obtained after segmentation meet the data input requirements of the first electrocardiogram analysis model, various implementations may be used here to segment the electrocardiogram data to be analyzed. Here, it is assumed that the electrocardiogram data to be analyzed is a signal sequence consisting of P frames of electrocardiogram data, each frame of electrocardiogram data including an L-lead electrocardiogram signal, wherein P and L are both positive integers.

Optionally, an average segmentation method may be used, starting from a first frame of the electrocardiogram data to be analyzed, to continuously acquire electrocardiogram data of F frames (F being a positive integer less than P) each time as an electrocardiogram data segment to be analyzed, and the electrocardiogram data segments continuously acquired twice do not overlap, until there is no electrocardiogram data which is not acquired in the electrocardiogram data to be analyzed or the number of frames of electrocardiogram data which is not acquired in the electrocardiogram data to be analyzed is greater than zero and less than F. Then remaining electrocardiogram data which is not acquired in the electrocardiogram data to be analyzed is acquired. Some electrocardiogram data is added to the finally acquired electrocardiogram data of greater than zero frame and less than F frames to form the electrocardiogram data of the F frames as the electrocardiogram data segment to be analyzed. With this average segmentation method, all the data in the electrocardiogram data to be analyzed can be obtained without missing data, so as to avoid that the incomplete data may lead to inaccurate subsequent generated heart disease diagnosis result information. In addition, there is no overlap between the electrocardiogram data segments to be analyzed, so that the number of electrocardiogram data to be analyzed input to the first electrocardiogram analysis model in the subsequent step 202 can be reduced, thereby reducing the calculation amount and improving the calculation speed.

Optionally, starting from the first frame of the electrocardiogram data to be analyzed, electrocardiogram data of F (F is a positive integer less than P) frames may also be continuously acquired each time as an electrocardiogram data segment to be analyzed by a sliding window segmentation method, and next, electrocardiogram data of F frames are acquired again after sliding W (W is a positive integer less than F) frames forward from a starting frame of electrocardiogram data acquired last time until there is no electrocardiogram data which is not acquired in the electrocardiogram data to be analyzed or the number of frames of electrocardiogram data which is not acquired in the electrocardiogram data to be analyzed is greater than zero and less than F. Then remaining electrocardiogram data which is not acquired in the electrocardiogram data to be analyzed is acquired, and some electrocardiogram data is added to the finally acquired electrocardiogram data of greater than zero frame and less than F frames to form electrocardiogram data of the F frames as the electrocardiogram data segment to be analyzed. With this sliding window segmentation method, not only can all the data in the electrocardiogram data to be analyzed be acquired, but also there is data overlap between two electrocardiogram data segments to be analyzed acquired adjacently, which can cover electrocardiogram data segments with various starting times. For example, each acquired electrocardiogram data segment to be analyzed may begin with a P-wave, PR interval, QRS complex, ST segment, T-wave, U-wave, or QT interval, etc. Since the types of electrocardiogram data segments that can be covered are more abundant, the types of electrocardiogram data segments to be analyzed input to the first electrocardiogram analysis model in the subsequent step 202 can be enriched, improving the accuracy of the generated heart disease diagnosis result information.

Each electrocardiogram data segment to be analyzed obtained by using the above-mentioned two segmentation methods includes F frames of electrocardiogram signals, and each frame of electrocardiogram signal includes L-lead electrocardiogram signal, so that the electrocardiogram data segment to be analyzed meets the requirements of the required input data of the first electrocardiogram analysis model.

In some alternative embodiments, the execution subject may also execute the following step 2011′ before step 2012:

Step 2011′, the electrocardiogram data to be analyzed is resampled so that a sampling frequency of the electrocardiogram data to be analyzed is a preset sampling frequency.

Here, the sampling frequency of the electrocardiogram data to be analyzed can be the preset sampling frequency by up-sampling or down-sampling the electrocardiogram data to be analyzed. Down-sampling requires decimation of the electrocardiogram data to be analyzed and up-sampling requires interpolation of the electrocardiogram data to be analyzed. It should be noted that various resampling methods now known or developed in the future may be employed, which is not particularly limited in the present disclosure.

By performing step 2011′, the sampling frequency of the electrocardiogram data to be analyzed is the preset sampling frequency. It is assumed that the preset sampling frequency is f Hertz, f being a positive integer, namely, there are f frames of electrocardiogram data per second in the electrocardiogram data to be analyzed. Then, when step 2012 is performed subsequently, if the average segmentation method or the sliding window segmentation method is used to segment the electrocardiogram data to be analyzed, each electrocardiogram data segment to be analyzed may include F frames of electrocardiogram data, wherein F may be a product of n and f, and n is a positive number. That is, each electrocardiogram data segment to be analyzed corresponds to electrocardiogram data having a duration of n seconds. Here, n seconds may be a length of time greater than an average human cardiac cycle preset by a skilled person with medical knowledge, so as to realize that in most cases a whole cardiac cycle may be covered in each electrocardiogram data segment to be analyzed, so as to improve the probability that each electrocardiogram data segment to be analyzed input to the first electrocardiogram analysis model in the subsequent step 202 covers the whole cardiac cycle, thereby improving the accuracy of the heart disease diagnosis result information generation.

Step 202, each electrocardiogram data segment to be analyzed is input into a pre-trained first electrocardiogram analysis model to obtain a heart disease suffering probability vector corresponding to the electrocardiogram data segment to be analyzed.

In this embodiment, the above-mentioned executive subject may, for each electrocardiogram data segment to be analyzed acquired in step 201, input the electrocardiogram data segment to be analyzed into the pre-trained first electrocardiogram analysis model to obtain the heart disease suffering probability vector corresponding to the electrocardiogram data segment to be analyzed. Here, the heart disease suffering probability vector is used to characterize a probability of suffering from each of K preset heart diseases. The first electrocardiogram analysis model is used to characterize a correspondence between the electrocardiogram data segment and the heart disease suffering probability vector, where K is a positive integer. Optionally, the heart disease suffering probability vector may be a K-dimensional vector, wherein K components in the K-dimensional vector are in one-to-one correspondence to K preset heart diseases, and each component is used for characterizing a probability of suffering from the corresponding preset heart disease of K preset heart diseases.

For example, the component of the heart disease suffering probability vector may have a value of 0-1, and the closer the value is to 1, the greater the possibility of suffering from the corresponding preset heart disease.

In practice, the heart disease can be divided into various types. Here, the K preset heart diseases may be K different heart diseases. Optionally, K is a positive integer greater than or equal to 2. In this way, the first electrocardiogram analysis model can output information on the disease suffering probability or degree of disease occurrence of two or more heart diseases, etc.

In some alternative embodiments, the K preset heart diseases may be K heart diseases selected from a preset heart disease set that may include, but is not limited to, the following heart diseases: sinus tachycardia (SNT), sinus bradycardia (SNB), premature atrial contraction (PAC), premature junctional contraction (PJC), premature ventricular contraction (PVC), supraventricular tachycardia (SVT), ventricular tachycardia (VT), atrial flutter (AFL), atrial fibrillation (AF), atrial escape (AE), junctional escape (JE), ventricular escape (VE), right bundle branch block (RBBB), sinus arrhythmia, sinus arrest, supraventricular premature beats, paired supraventricular premature beats, bigeminy coupled rhythm of supraventricular premature beats, trigeminy of supraventricular premature beats, ventricular premature beats, paired ventricular premature beats, bigeminy coupled rhythm of ventricular premature beats, trigeminy of ventricular premature beats, supraventricular escape beats, Wolf-Parkinson-White (WPW) syndrome, ventricular flutter, ventricular fibrillation, ventricular escape, first degree atrio-ventricular block (IVAB), secondary degree atrio-ventricular block (IIVAB), third degree atrio-ventricular block (IIIVAB), intra-ventricular block (IVB), left bundle branch block (LBBB), complete right bundle branch block (CRBBB), conduction block in left forearm, left ventricular hypertrophy, right ventricular hypertrophy, left atrial hypertrophy and right atrial hypertrophy. As an example, the first electrocardiogram analysis model may be formulated in advance by a skilled person, after performing statistical analysis on electrocardiogram data segments of patients diagnosed with different heart diseases in K preset heart diseases on the basis of a large number of practices, calculating the data of different leads of electrocardiogram data at different time points in the electrocardiogram data segments and obtaining a calculation formula for the disease suffering probability of the K different preset heart diseases.

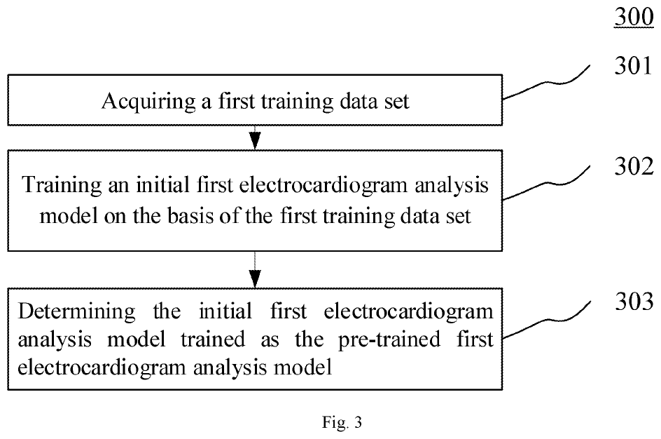

In some alternative embodiments, the first electrocardiogram analysis model may also be pre-trained by a first training step 300 as shown in FIG. 3, where the first training step 300 may include the following steps 301 to 303:
Here, the executive subject of the first training step may be the same as or different from the executive subject of the electrocardiogram analysis method described above. If the executive subject of the first training step is the same as the executive subject of the above-mentioned electrocardiogram analysis method, the executive subject of the first training step may store model structure information and the parameter values of model parameters of the trained first electrocardiogram analysis model locally in the above-mentioned executive subject after the first electrocardiogram analysis model is trained. If the executive subject of the first training step is different from the executive subject of the above-mentioned electrocardiogram analysis method, the executive subject of the first training step may send the model structure information and the parameter values of the model parameters of the trained first electrocardiogram analysis model to the executive subject of the above-mentioned electrocardiogram analysis method after the first electrocardiogram analysis model is trained.

Step 301, a first training data set is acquired.

Here, first training data may include a sample electrocardiogram data segment and a corresponding labeled heart disease suffering probability vector. The labeled heart disease suffering probability vector in the first training data is used to indicate a probability of a person, whom the sample electrocardiogram data segment in the first training data corresponds to and on whom collection is performed, suffering from each of the above-mentioned K preset heart diseases.

Optionally, the first training data set may be obtained by the following manners:
first, a set of sample electrocardiogram data obtained by performing electrocardiogram examination on different subjects is acquired.

Secondly, by a skilled person with professional medical knowledge, according to sample electrocardiogram data obtained by performing electrocardiogram examination on the same subject in a sample electrocardiogram data set, it is diagnosed whether the subject suffers from the above-mentioned K preset heart diseases, all the sample electrocardiogram data of the subject is labeled according to the diagnosis result of the subject, so as to determine the probability of the subject suffering from each of K preset heart diseases, and then a labeled heart disease suffering probability vector corresponding to each sample electrocardiogram data can be obtained.

Then, each sample electrocardiogram data in the sample electrocardiogram data set can be segmented, and sample electrocardiogram data segments can be obtained. Reference is made to the related description of the above step 2012 for details of how to perform the segmentation, which will not be repeated here.

Optionally, prior to segmenting each sample electrocardiogram data in the sample electrocardiogram data set, each sample electrocardiogram data in the sample electrocardiogram data set is resampled such that the sampling frequency of each sample electrocardiogram data is a preset sampling frequency. Then, each sample electrocardiogram data after the resampling is segmented so that the sampling frequency of each obtained sample electrocardiogram data segment is also the preset sampling frequency. With regard to the specific resampling method, reference can be made to the relevant description in the above step 2011′, which will not be repeated here.

Finally, the first training data is generated using the obtained sample electrocardiogram data segment and the labeled heart disease suffering probability vector corresponding to the sample electrocardiogram data from which the sample electrocardiogram data segment is derived, and then the first training data set is obtained.

Step 302, an initial first electrocardiogram analysis model is trained on the basis of the first training data set.

Here, the initial first electrocardiogram analysis model may be trained on the basis of the first training data set using various machine learning methods.

Here, the initial first electrocardiogram analysis model may be various machine learning models. For example, the initial first electrocardiogram analysis model may be an artificial neural network (ANN), a deep learning (DL) model, a support vector machine (SVM), a random forest (RF), a decision tree (DT), a linear regression (LR), a logistic regression (LR), a Poisson regression (PR), a ridge regression, a Lasso regression, a k-nearest neighbor (KNN), a linear discriminant analysis (LDA), and a logarithmic linear model (LLM), or the like.

Optionally, the initial first electrocardiogram analysis model may be a deep learning model and may include a convolutional layer, a batch normalization layer, an activation function layer, a dropout layer, a fully connected layer, and a pooling layer.

In particular, step 302 may be performed as follows:
Firstly, the sample electrocardiogram data segments in the first training data of the first training data set are input into the initial first electrocardiogram analysis model to obtain the corresponding heart disease suffering probability vector.

It should be noted here that for one first training data in the first training data set each time, or for each first training data in a batch of first training data, the above-mentioned sample electrocardiogram data segments in each first training data are input into the initial first electrocardiogram analysis model to obtain the corresponding heart disease suffering probability vector.

Secondly, the difference between the resulting heart disease suffering probability vector and the labeled heart disease suffering probability vector in the corresponding first training data is calculated.

Here, various difference calculation methods (or loss functions) may be used to calculate the difference between the resulting heart disease suffering probability vector and the labeled heart disease suffering probability vector in the corresponding first training data. For example, the above-mentioned loss function may be an L1-norm loss function (also referred to as Least Absolute Deviations (LAD), or least Absolute Error (LAE)), an L2-norm loss function (also referred to as Least Squared Error, LSE), a zero-one loss function, an absolute value loss function, a logarithmic loss function, a squared loss function, an exponential loss function, a Hinge loss function, a perceptual loss function, a cross-entropy loss function), or the like. Finally, the model parameters of the initial first electrocardiogram analysis model are adjusted on the basis of the obtained differences until a preset training end condition is met.

Here, various parameter optimization methods may be employed to adjust the model parameters of the initial first electrocardiogram analysis model on the basis of the resulting differences. For example, the following gradient descent (GD) optimization algorithms may be employed: batch gradient descent (BGD), mini-batch gradient descent (MBGD), stochastic gradient descent (SGD), gradient descent with momentum (GDM), Nesterov accelerated gradient (NAG), RMSprop (Root Mean Square Prop) algorithm, adaptive moment estimation (Adam) algorithm, etc.

As another example, a newton's method, a quasi-Newton method, a conjugate gradient method, a heuristic optimization method, and the like may also be employed, which is not particularly limited in the present disclosure.

Here, the preset training end condition may be various preset conditions for determining model convergence. For example, the preset training end conditions may include at least one of:
A condition 1, the number of times of performing step 302 is greater than or equal to a preset number of times.
A condition 2, step 302 is performed for more than a preset training duration.
A condition 3, the difference obtained in step 302 is less than a preset difference threshold.
A condition 4, prior to step 302, a verification data set is acquired in advance. Verification data in the verification data set includes a verified electrocardiogram data segment and a corresponding labeled heart disease suffering probability vector. Also, a subject to which the verification data set corresponds is completely different from the subject to which the first training data set corresponds. The verified electrocardiogram data segment in the verification data set may be obtained in the same or similar manner as that of acquiring the first training data set described in step 301, which will not be described in detail herein. Then, the difference between the difference obtained in this step 302 and the difference calculated when the model parameters of the initial first electrocardiogram analysis model were adjusted last time is calculated. The condition 4 is that the difference calculated above is less than a preset loss function difference threshold. That is, the loss function of the initial first electrocardiogram analysis model no longer decreases or decreases very little in magnitude in the verification data set.

The model parameters of the initial first electrocardiogram analysis model are optimized via step 302.

Step 303, the trained initial first electrocardiogram analysis model is determined as the pre-trained first electrocardiogram analysis model.

Through step 301 to step 303, the first electrocardiogram analysis model can be obtained in which the model parameters are optimized by training using the first training data set.

Step 203, heart disease diagnosis result information of the target user is generated on the basis of the heart disease suffering probability vector corresponding to each electrocardiogram data segment to be analyzed.

Each electrocardiogram data segment to be analyzed acquired in step 201 is obtained on the basis of electrocardiogram data of performing electrocardiogram examination on the target user, and it is assumed that J (J is a positive integer) electrocardiogram data segments to be analyzed are acquired in step 201. After step 202, a heart disease suffering probability vector corresponding to each electrocardiogram data segment to be analyzed in the J electrocardiogram data segments to be analyzed can be obtained, namely, J heart disease suffering probability vectors can be obtained. Each heart disease suffering probability vector is used for characterizing the probability of the target user suffering from each of K preset heart diseases. Each heart disease suffering probability vector may be a K-dimensional vector, wherein each component may be used to characterize the probability of suffering from one preset heart disease.

Here, the executive subject may generate heart disease diagnosis result information of the target user on the basis of the J K-dimensional heart disease suffering probability vectors obtained in step 202 in various manners according to the needs of a specific application scenario.

Here, the heart disease diagnosis result information may be in various forms. For example, a text, an image, and voice data may be included, but are not limited thereto.

Here, the heart disease diagnosis result information may be various information related to the heart disease diagnosis. The heart disease diagnosis result information may be used to indicate that a certain preset heart disease is diagnosed, or that a certain preset heart disease is not diagnosed, or may also be used to indicate the degree of suffering from a certain preset heart disease. Here, the degree information may be represented by numerical values or by words. For example, the degree information may be a degree value of 0-1. The degree information may also include, for example, “the risk of suffering from a certain preset heart disease is very high”, “the risk of suffering from a certain preset heart disease is high”, “the risk of suffering from a certain preset heart disease is low”, “the risk of suffering from a certain preset heart disease is very low”, etc.

In some alternative embodiments, step 203 may be performed in the following manners:

for each preset heart disease, a first diagnosis result information generating operation is performed. The first diagnosis result information generating operation may include steps 2031A to 2033A as shown in FIG. 2B:
Step 2031A, a heart disease suffering probability of the target user suffering from the preset heart disease is determined according to a component corresponding to the preset heart disease in the heart disease suffering probability vector corresponding to each electrocardiogram data segment to be analyzed.

Here, the heart disease suffering probability of the target user suffering from the preset heart disease can be determined in various ways. It is assumed that i is a positive integer of 1-K, and Dᵢ is the ith preset heart disease of the K preset heart diseases. In step 201, J (j is a positive integer from 1 to J) electrocardiogram data segments to be analyzed are acquired, SEⱼ is the jth electrocardiogram data segment to be analyzed therein, VPⱼ is a heart disease suffering probability vector corresponding to the electrocardiogram data segment to be analyzed SEⱼ, and VPji is the ith dimensional component of VPⱼ. Here, with regard to the preset heart disease Dᵢ, the heart disease suffering probability of the target user suffering from the preset heart disease may be determined according to the ith dimensional component in the heart disease suffering probability vector VPⱼ corresponding to each electrocardiogram data segment to be analyzed SEⱼ (j is a positive integer of 1-J) in the J electrocardiogram data segments to be analyzed.

Optionally, a mean value of components corresponding to the preset heart disease in the heart disease suffering probability vector corresponding to each electrocardiogram data segment to be analyzed may be determined as the heart disease suffering probability of the target user suffering from the preset heart disease. In this way, the heart disease suffering probability vectors corresponding to the electrocardiogram data segments to be analyzed can be comprehensively considered to improve the comprehensiveness of the generation of diagnosis result information. Continuing with the above-mentioned assumption, here, for the preset heart disease Dᵢ, the following formula can be used to express the heart disease suffering probability Prbᵢ of the target user suffering from the preset heart diseases Dᵢ:

$${Prb}_{i} = \frac{1}{J} {\sum }_{j = 1}^{J} {VP}_{j , i} \qquad ( 1 )$$

Or optionally, the components corresponding to the preset heart disease in the heart disease suffering probability vector corresponding to each electrocardiogram data segment to be analyzed may be ranked in an order from low to high, and a component ranked in a preset quantile may be determined as the heart disease suffering probability of the target user suffering from the preset heart disease. Continuing with the above-mentioned assumption, here, VP_{j,i} (j is a positive integer of 1-J) is first ranked in an order from low to high, VP_{j′,i} being ranked at the preset quantile, j′ being a positive integer of 1-J. Here, the heart disease suffering probability Prbᵢ of the target user suffering from the preset heart disease Dᵢ may be VP_{j′,i}.

Optionally, step 2031A may also be performed as follows:

first, the components, which correspond to the preset heart disease, of the heart disease suffering probability vector corresponding to each electrocardiogram data segment to be analyzed may be ranked from low to high. Continuing with the above-mentioned assumption, it is intended here that for a preset heart disease Dᵢ, VP_{j,i} (j is a positive integer of 1-J) is ranked from low to high.

Then, it can be determined whether the current application scenario is a less false positive scenario or less false negative scenario.

In practice, electrocardiogram examination of a human body is generally divided into a less false positive scenario and a less false negative scenario. In the less false positive scenario, the diagnosis of a patient who does not suffer from a heart disease as suffering from a heart disease should be minimized, i.e. determined/diagnosed as not suffering from the heart disease as possible. The less false positive scenario may be, for example, a diagnostic scenario, i.e. a scenario where a patient goes to a hospital to see a doctor after suffering from heart disease-related symptoms and a physician prescribes an electrocardiogram examination. In the less false negative scenario, determining subjects suffering from the heart disease as not suffering from the heart disease should be minimized, i.e. determined/screened as suffering from the heart disease. The less false negative scenario may be, for example, a screening scenario, such as examining an electrocardiogram during a routine physical examination.

The manner in which the heart disease suffering probability of the target user suffering from the preset heart disease is determined may be correspondingly different for different application scenarios.

If it is determined that the current application scenario is a less false positive scenario, the corresponding requirement is that the target user is diagnosed as not suffering from the preset heart disease as far as possible, i.e. the heart disease suffering probability of the target user suffering from the preset heart disease should also be as low as possible. Here, the minimum value of the components corresponding to the preset heart disease in the heart disease suffering probability vector corresponding to each electrocardiogram data segment to be analyzed or the component ranked in a preset lower probability quantile can be determined as the heart disease suffering probability of the target user suffering from the preset heart disease. Since the components corresponding to the preset heart disease in the heart disease suffering probability vector corresponding to each electrocardiogram data segment to be analyzed are ranked in the order from low to high, according to the above-mentioned ranking result, the component values corresponding to the preset heart disease in the heart disease suffering probability vector corresponding to the electrocardiogram data segment to be analyzed at a position with a smaller quantile are correspondingly smaller, and thus the determined heart disease suffering probability of the target user suffering from the preset heart disease is also smaller. Here, the preset lower probability quantile may be a quantile of less than 50%. For example, the preset lower probability quantile may be a quantile of 25%, namely, the heart disease suffering probability of the target user suffering from the preset heart disease is a relatively small or minimum component corresponding to the preset heart disease in the heart disease suffering probability vector corresponding to the electrocardiogram data segment to be analyzed in the components corresponding to the preset heart disease in the heart disease suffering probability vector corresponding to each electrocardiogram data segment to be analyzed. For ease of understanding, continuing with the above-mentioned assumption, the probability Prbᵢ of the target user suffering from the preset heart disease Dᵢ can be expressed as follows with a formula:

$${Prb}_{i} = {min}_{j} ( {VP}_{j , i} ) \qquad ( 2 )$$

$$or$$

$${Prb}_{i} = {VP}_{{j}^{s} , i} \qquad ( 3 )$$

wherein the ranking of VPis; in VP_{j,i} (j and is being a positive integer of 1-J) is at the preset lower probability quantile.

If it is determined that the current application scenario is a less false negative scenario, the corresponding requirement is to diagnose the target user as suffering from the preset heart disease as far as possible, i.e. the heart disease suffering probability of the target user suffering from the preset heart disease should also be as large as possible. Here, the maximum value of the components corresponding to the preset heart disease in the heart disease suffering probability vector corresponding to each electrocardiogram data segment to be analyzed or the component ranked at a preset higher probability quantile can be determined as the heart disease suffering probability of the target user suffering from the preset heart disease. Since the components corresponding to the preset heart disease in the heart disease suffering probability vector corresponding to each electrocardiogram data segment to be analyzed are ranked in the order from low to high, according to the above-mentioned ranking result, the component values corresponding to the preset heart disease in the heart disease suffering probability vector corresponding to the electrocardiogram data segment to be analyzed at a position with a larger quantile are correspondingly larger, and thus the determined heart disease suffering probability of the target user suffering from the preset heart disease is also larger. Here, the component of the preset higher probability quantile may be a quantile of larger than 50%. For example, the preset higher probability quantile may be a quantile of 75%, namely, the heart disease suffering probability of the target user suffering from the preset heart disease is a relatively greater or maximum component corresponding to the preset heart disease in the heart disease suffering probability vector corresponding to the electrocardiogram data segment to be analyzed in the components corresponding to the preset heart disease in the heart disease suffering probability vector corresponding to each electrocardiogram data segment to be analyzed. For ease of understanding, continuing with the above-mentioned assumption, the probability Prbᵢ of the target user suffering from the preset heart disease Dᵢ can be expressed as follows with a formula:

$${Prb}_{i} = {min}_{j} ( {VP}_{j , i} ) \qquad ( 4 )$$

$$or$$

$${Prb}_{i} = {VP}_{{j}^{b} , i} \qquad ( 5 )$$

wherein the ranking of VPⱼ_{^{b}}_{,i} in VP_{j,i} (j and j^{b} being a positive integer of 1-J) is at the preset higher probability quantile.

Step 2032A, heart disease diagnosis result information corresponding to the heart disease suffering probability of the target user suffering from the preset heart disease is determined according to a correspondence between a disease suffering probability range and heart disease diagnosis result information corresponding to the preset heart disease.

Here, the correspondence between a disease suffering probability range and heart disease diagnosis result information corresponding to each of the K preset heart diseases may be set in advance. Then, in step 2032A, a disease suffering probability range to which the heart disease suffering probability of the target user suffering from the preset heart disease belongs may be found first in the correspondence between a disease suffering probability range and heart disease diagnosis result information corresponding to the preset heart disease, and then heart disease diagnosis result information corresponding to the found disease suffering probability range is determined as the heart disease diagnosis result information corresponding to the heart disease suffering probability of the target user suffering from the preset heart disease.

Optionally, the correspondence between a disease suffering probability range and heart disease diagnosis result information corresponding to the preset heart disease may include at least one of: a first correspondence and a second correspondence. wherein:
the first correspondence is used for characterizing that a first disease suffering probability range corresponds to first diagnosis result information indicating that the preset heart disease is not diagnosed, wherein the first disease suffering probability range is less than a disease suffering probability threshold corresponding to the preset heart disease. By using the first correspondence, first diagnosis result information that the target user is not diagnosed with the preset heart disease can be generated in the case where the heart disease suffering probability of the target user suffering from the preset heart disease obtained in step 2031A is less than the disease suffering probability threshold corresponding to the preset heart disease.

The second correspondence is used for characterizing that a second disease suffering probability range corresponds to second diagnosis result information indicating that the preset heart disease is diagnosed, wherein the second disease suffering probability range is greater than or equal to a disease suffering probability threshold corresponding to the preset heart disease. By using the second correspondence, second diagnosis result information that the target user is diagnosed with the preset heart disease can be generated in the case where the heart disease suffering probability of the target user suffering from the preset heart disease obtained in step 2031A is greater than or equal to the disease suffering probability threshold corresponding to the preset heart disease.

Step 2033A, heart disease diagnosis result information of the target user suffering from the preset heart disease is generated using the determined heart disease diagnosis result information. In some alternative embodiments, step 203 may also be performed as follows: for each preset heart disease, a second diagnosis result information generating operation is performed. The second diagnosis result information generating operation may include steps 2031B to 2034B as shown in FIG. 2C.

Step 2031B, a set of disease suffering probability ranges corresponding to the preset heart disease is acquired.

Here, a corresponding disease suffering probability range set may be preset for each of the K preset heart diseases. It is to be understood that the disease suffering probability ranges corresponding to different preset heart diseases may be the same or different, and that there is no overlapping range between any two disease suffering probability ranges in the set of disease suffering probability ranges corresponding to the same preset heart disease.

Here, it is assumed that i is a positive integer from 1 to K, and Dᵢ is the ith preset heart disease among the K preset heart diseases. Step 2031B refers to, with regard to the preset heart disease Dᵢ, acquiring a set SCPᵢ of disease suffering probability ranges corresponding to Dᵢ, wherein the SCPᵢ includes U disease suffering probability ranges, u1 and u2 are respectively positive integers of 1-U, u1 is not equal to u2,scpᵤ₁ and scpᵤ₂ are respectively the u1th and u2th disease suffering probability ranges in the SCPi, and there is no overlapping range between scpᵤ₁ and scpᵤ₂. Step 2032B, for each acquired disease suffering probability range, a proportion of data segments corresponding to the disease suffering probability range is determined.

Here, the proportion of data segments corresponding to the disease suffering probability range is a proportion of the number of components belonging to the disease suffering probability range in the components corresponding to the preset heart disease in the heart disease suffering probability vector corresponding to each electrocardiogram data segment to be analyzed divided by the number of the electrocardiogram data segments to be analyzed.

Step 2032B refers to determining a proportion SRᵤ of data segments corresponding to scpᵤ for each disease suffering probability range scpᵤ (u is a positive integer of 1-U). It is assumed that J electrocardiogram data segments to be analyzed are acquired in step 201, j being a positive integer from 1 to J, and SEⱼ is the jth electrocardiogram data segment to be analyzed. VPⱼ is the heart disease suffering probability vector corresponding to the electrocardiogram data segment to be analyzed SEⱼ, and VP_{j,i} is the ith dimensional component of VPⱼ. Here, the number Num_{i,u}of the heart disease suffering probability vectors VPⱼ corresponding to each electrocardiogram data segment to be analyzed SEⱼ can be determined first, wherein, in the heart disease suffering probability vectors VPⱼ, the component VP_{j,i} corresponding to the preset heart disease Dᵢ belongs to the disease suffering probability range scpᵤ. And, the proportion SRᵤ of data segments corresponding to scpᵤ refers to a proportion obtained by dividing Num_{i,u} u by J.

Step 2033B, according to a correspondence between a disease suffering probability range and heart disease diagnosis result information corresponding to the preset heart disease, heart disease diagnosis result information corresponding to the disease suffering probability range with the largest proportion of the corresponding data segments is determined.

Here, it is necessary to first determine the disease suffering probability range scpmax with the largest proportion of the corresponding data segments in each disease suffering probability range scpᵤ acquired in step 2031B, and then determine the heart disease diagnosis result information corresponding to the disease suffering probability range scpmax with the largest proportion of the corresponding data segments according to the correspondence between a disease suffering probability range and heart disease diagnosis result information corresponding to the preset heart disease.

Step 2034B, heart disease diagnosis result information of the target user suffering from the preset heart disease is generated using the determined heart disease diagnosis result information. With this alternative embodiment, for each preset heart disease Dᵢ, the heart disease diagnosis result information corresponding to the disease suffering probability range scpₘₐₓ with the largest proportion of the corresponding data segments in each disease suffering probability range scpu corresponding to the preset heart disease Dᵢ is used to generate the heart disease diagnosis result information of the target user suffering from the preset heart disease Dᵢ.

Due to the inconsistency, instability, uncertainty and other factors in the onset time, period and frequency of different types of heart diseases, among the electrocardiogram data segments to be analyzed obtained by performing electrocardiogram examination on the target user, in most cases, it may not be possible to determine whether the target user suffers from each preset heart disease according to one electrocardiogram data segment to be analyzed. However, if it can be determined that the target user suffers from a certain preset heart disease on the basis of more than a certain percentage of the electrocardiogram data segments to be analyzed among the electrocardiogram data segments to be analyzed, it can be determined that the target user suffers from the preset heart disease. Thus, in some alternative embodiments, step 203 may include steps 2031C and 2032C as shown in FIG. 2D:
Step 2031C, for each preset heart disease, the preset heart disease is labeled as a diagnosed heart disease in response to determining that a proportion of diagnosis electrocardiogram data segments corresponding to the preset heart disease is not less than a diagnosis proportion threshold corresponding to that preset heart disease.

Here, the proportion of the diagnosis electrocardiogram data segments corresponding to the preset heart disease is a proportion of the number of the diagnosis electrocardiogram data segments corresponding to the preset heart disease divided by the total number (i.e. J) of the electrocardiogram data segments to be analyzed, and the number of the diagnosis electrocardiogram data segments corresponding to the preset heart disease is the number of the components corresponding to the preset heart disease in the heart disease suffering probability vector corresponding to each electrocardiogram data segment to be analyzed, which are greater than a disease suffering probability threshold corresponding to the preset heart disease. That is, if the component corresponding to the preset heart disease in the heart disease suffering probability vector corresponding to a certain electrocardiogram data segment to be analyzed is greater than the disease suffering probability threshold corresponding to the preset heart disease, it is indicated that the electrocardiogram data segment to be analyzed acquires the moment when the target user has the preset heart disease, it can be determined that the target user suffers from the preset heart disease according to the electrocardiogram data segment to be analyzed. If the electrocardiogram data segments to be analyzed that exceed the diagnosis proportion threshold present in each electrocardiogram data segment to be analyzed determine that the target user suffers from the preset heart disease, the preset heart disease may be labeled as diagnosed heart disease.

For example, the diagnosis proportion threshold corresponding to each preset heart disease may be a value of greater than or equal to 0.5 but less than or equal to 1.

Step 2032C, heart disease diagnosis result information indicating that the target user is diagnosed with a diagnosed the heart disease among the preset heart diseases, which is labeled as a diagnosed heart disease, is generated.

With this alternative embodiment, the diagnosis result information for determining that the target user suffers from the preset heart disease can be generated in a case where it can be determined that the target user suffers from the preset heart disease on the basis of the electrocardiogram data segments to be analyzed which exceed the diagnosis proportion threshold among the electrocardiogram data segments to be analyzed.

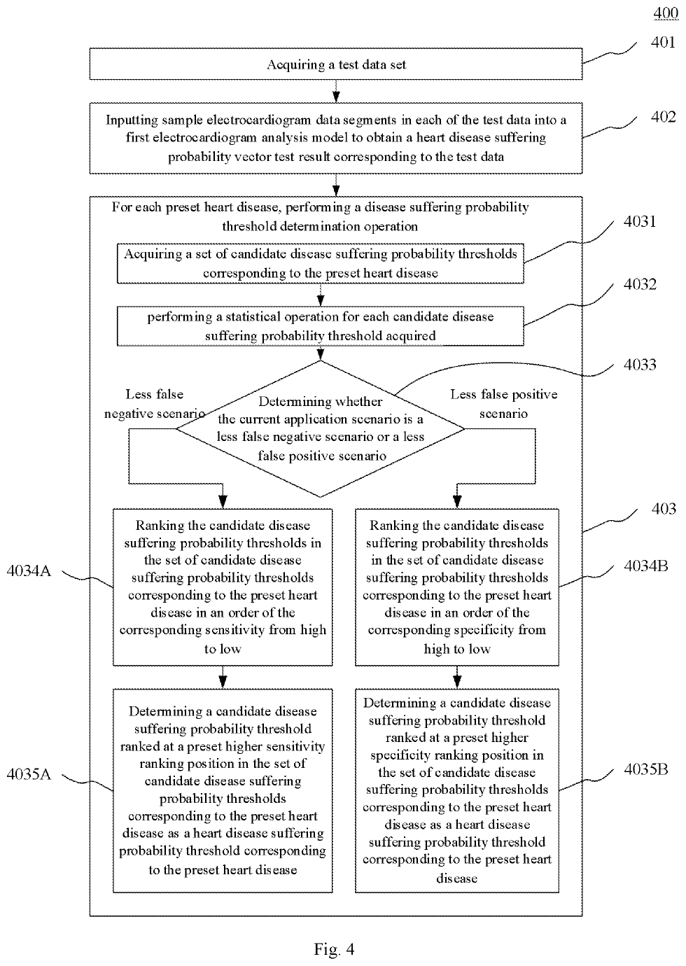

In some alternative embodiments, the disease suffering probability threshold corresponding to each preset heart disease may be obtained by a disease suffering probability threshold determination step 400 as shown in FIG. 4, and the disease suffering probability threshold determination step 400 may include the following steps 401 to 403:
Step 401, a test data set is acquired.

Here, test data may include a sample electrocardiogram data segment and a labeled heart disease suffering probability vector.

The labeled heart disease suffering probability vector in the test data can be used to indicate a probability of a person, whom the sample electrocardiogram data segment in the test data corresponds to and on whom collection is performed, suffering from each preset heart disease.

Step 402, sample electrocardiogram data segments in each test data are input into the first electrocardiogram analysis model to obtain a heart disease suffering probability vector test result corresponding to the test data.

Step 403, for each preset heart disease, a disease suffering probability threshold determination operation is performed.

Here, the disease suffering probability threshold determination operation may include the following steps 4031, 4032, 4033, 4034A, 4035A, 4034B, and 4035B:
Step 4031, a set of candidate disease suffering probability thresholds corresponding to the preset heart disease is acquired.

Here, it is assumed that i is a positive integer from 1 to K, and Dᵢ is the ith preset heart disease among K preset heart diseases. Here, the set ThPᵢ of candidate disease suffering probability thresholds corresponding to the preset heart disease Dᵢ can be obtained. Here, the candidate disease suffering probability threshold set ThPᵢ may include at least two candidate disease suffering probability thresholds. The set of candidate disease suffering probability thresholds corresponding to different preset heart diseases may be the same or different. For example, a set ThP₁ of candidate disease suffering probability thresholds corresponding to a preset heart disease D₁ may be {0.1, 0.2, 0.25, 0.3, 0.4, 0.5, 0.55, 0.6, 0.7, 0.8, 0.9, 0.95}, and a set ThP₂ of candidate disease suffering probability thresholds corresponding to a preset heart disease D2 may be {0.15, 0.35, 0.42, 0.56, 0.62, 0.7, 0.75, 0.86, 0.9, 0.97}.

Step 4032, for each candidate disease suffering probability threshold acquired, a statistical operation is performed.

Here, it is assumed that the set ThPᵢ of candidate disease suffering probability thresholds corresponding to the preset heart disease Dᵢ includes Q candidate disease suffering probability thresholds, Q being a positive integer. ThPᵢ[q] represents the qth candidate disease suffering probability threshold in the candidate disease suffering probability threshold set ThPᵢ. Here q is a positive integer of 1-Q.

Here, it means that a corresponding statistical operation is performed on each candidate disease suffering probability threshold ThPᵢ[q] in the set ThPᵢ of candidate disease suffering probability thresholds corresponding to the preset heart disease Dᵢ. Here, the statistical operation may be performed as follows:

According to whether a vector component corresponding to the preset heart disease Dᵢ in a heart disease suffering probability vector test result corresponding to each test data is greater than the candidate disease suffering probability threshold ThPᵢ[q], and whether a vector component corresponding to the preset heart disease in a labeled heart disease suffering probability vector in the corresponding test data is greater than the candidate disease suffering probability threshold ThPᵢ[q], counting sensitivity (namely, a true positive rate) and specificity (namely, a true negative rate) corresponding to the preset heart disease Dᵢ and the candidate disease suffering probability threshold ThPᵢ[q]. The specific formula can be expressed as follows:

$${SE}_{i , q} = \frac{{TP}_{i , q}}{{TP}_{i , q} + {FN}_{i , q}} \qquad ( 6 )$$

$${SP}_{i , q} = \frac{{TN}_{i , q}}{{TN}_{i , q} + {FP}_{i , q}} \qquad ( 7 )$$

wherein SE_{i,q} and SP_{i,q} are the sensitivity and specificity respectively statistically obtained corresponding to the preset heart disease Dᵢ and the candidate disease suffering probability threshold ThPᵢ[q]. TP_{i,q}, FN_{i,q}, TN_{i,q} and FP_{i,q} are respectively the number of true positive results, the number of false negative results, the number of true negative results and the number of false positive results obtained by diagnosing the preset heart disease Dᵢ using each test data in the test data set according to the candidate disease suffering probability threshold ThPᵢ[q].

Here, the term “true positive” means that the components corresponding to the preset heart disease Dᵢ in the labeled heart disease suffering probability vector in the test data and the corresponding heart disease suffering probability vector test result are greater than the candidate disease suffering probability threshold Thi[q], that is, according to the candidate disease suffering probability threshold ThPᵢ[q], both the test on the test electrocardiogram data segment in the test data and the actual labeling indicate that the target user suffers from the preset heart disease Dᵢ. Namely, according to the candidate disease suffering probability threshold ThPᵢ[q], the test result of testing the preset heart disease Dᵢ on the basis of the test data is a true positive result.

Here, the term “false negative” means that the component corresponding to the preset heart disease Dᵢ in the labeled heart disease suffering probability vector in the test data is greater than the candidate disease suffering probability threshold ThPᵢ[q], namely, the target user suffers from the preset heart disease Dᵢ according to the actual labeling. However, the component corresponding to the preset heart disease Dᵢ in the heart disease suffering probability vector test result corresponding to the test data is not greater than the candidate disease suffering probability threshold ThPᵢ[q], that is to say, according to the candidate disease suffering probability threshold ThPᵢ[q], the test on the test electrocardiogram data segment in the test data indicates that the target user does not suffer from the preset heart disease Dᵢ, but indicates that the target user suffers from the preset heart disease Dᵢ according to the actual labeling. Namely, according to the candidate disease suffering probability threshold Thᵢ[q], the test result of testing the preset heart disease Dᵢ on the basis of the test data is a false negative result.

Here, the term “true negative” means that the components corresponding to the preset heart disease Dᵢ in the labeled heart disease suffering probability vector in the test data and the heart disease suffering probability vector test result corresponding to the test data are not greater than the candidate disease suffering probability threshold ThPᵢ[q], that is, according to the candidate disease suffering probability threshold ThPᵢ[g], both the test on the electrocardiogram data segment in the test data and the actual labeling indicate that the target user does not suffer from the preset heart disease Dᵢ. Namely, according to the candidate disease suffering probability threshold Thᵢ[q], the test result of testing the preset heart disease Dᵢ on the basis of the test data is a true negative result.

Here, the term “false positive” means that the component corresponding to the preset heart disease Dᵢ in the labeled heart disease suffering probability vector in the test data is not greater than the candidate disease suffering probability threshold Thᵢ[q], namely, the target user does not suffer from the preset heart disease Dᵢ according to the actual labeling. However, the component corresponding to the preset heart disease Dᵢ in the heart disease suffering probability vector test result of the test data is greater than the candidate disease suffering probability threshold ThPᵢ[q], that is to say, according to the candidate disease suffering probability threshold Thᵢ[q], the test on the test electrocardiogram data segment in the test data indicates that the target user suffers from the preset heart disease Dᵢ, but indicates that the target user does not suffer from the preset heart disease Dᵢ according to the actual labeling. Namely, according to the candidate disease suffering probability threshold ThPᵢ[q], the test result of testing the preset heart disease Dᵢ on the basis of the test data is a false positive result.

The sensitivity SEij and specificity SP_{i,j} corresponding to each candidate disease suffering probability threshold ThPᵢ[q] in the set ThPᵢ of candidate disease suffering probability thresholds corresponding to the preset heart disease Dᵢ can be obtained via step 4032.

Step 4033, whether the current application scenario is a less false negative scenario or a less false positive scenario is determined.

In practice, electrocardiogram examination of a human body is generally divided into less false negative scenarios (e.g. screening scenarios, i.e. electrocardiogram examination during routine physical examination) and less false positive scenarios (e.g. diagnostic scenarios, i.e. electrocardiogram examination after a patient goes to a hospital to see a doctor due to symptoms related to the heart disease). The heart disease suffering probability threshold corresponding to each preset heart disease may be different for different application scenarios.

If it is determined that the current application scenario is a less false negative scenario, the corresponding requirement is to screen out more true positive cases (i.e. corresponding to a higher true positive rate, screening real patients as patients with a higher probability), and screen out false negative cases as few as possible (i.e. corresponding to a lower false negative rate, screening real patients as healthy people with a lower probability), step 4034A may be performed.

If it is determined that the current application scenario is a less false positive scenario, the corresponding requirement is to diagnose more true negative cases (i.e. corresponding to a higher true negative rate, diagnosing a real healthy person as a healthy person with a higher probability), and to diagnose false positive cases as few as possible (i.e. corresponding to a lower false positive rate, diagnosing a real healthy person as a patient with a lower probability), step 4034B may be performed.

Step 4034A, candidate disease suffering probability thresholds in the set of candidate disease suffering probability thresholds corresponding to the preset heart disease are ranked in an order of the corresponding sensitivity from high to low.

That is, if it is determined in step 4033 that the current application scenario is a less false negative scenario, in step 4034A, the candidate disease suffering probability thresholds ThPᵢ[q] in the set ThPᵢ of candidate disease suffering probability thresholds corresponding to the preset heart disease Dᵢ can be ranked in the order of the sensitivities SE_{i,j} corresponding to the candidate disease suffering probability thresholds ThPᵢ[q] from high to low to obtain a first ranking result of each candidate disease suffering probability threshold ThPᵢ[q] in the candidate disease suffering probability threshold set ThPᵢ.

Step 4035A may be performed after execution of step 4034A.

Step 4035A, a candidate disease suffering probability threshold ranked at a preset higher sensitivity ranking position in the set of candidate disease suffering probability thresholds corresponding to the preset heart disease is determined as a heart disease suffering probability threshold corresponding to the preset heart disease.

That is, the candidate disease suffering probability threshold ThPᵢ[q] ranked at the preset higher sensitivity ranking position in the set ThPᵢ of candidate disease suffering probability thresholds corresponding to the preset heart disease Dᵢ is determined as the heart disease suffering probability threshold corresponding to the preset heart disease Dᵢ. The preset higher sensitivity ranking position may be a position ranked in the top first preset percentage in the candidate disease suffering probability threshold set ThPᵢ, such as the last one ranked in first 5%. The preset higher sensitivity ranking position may also be a top first preset position ranked in the candidate disease suffering probability threshold set ThPᵢ, such as ranking in the third.

Here, the sensitivity SE_{i,j} corresponding to the candidate disease suffering probability threshold ThPᵢ[q] ranked at the preset higher sensitivity ranking position in the candidate disease suffering probability threshold set ThPᵢ is relatively high. The candidate disease suffering probability threshold ThPᵢ[q] corresponding to the relatively high sensitivity SE_{i,j} is determined as the heart disease suffering probability threshold corresponding to the preset heart disease Dᵢ. A relatively high true positive rate and a relatively low false negative rate can be achieved in the process of generating the heart disease diagnosis result information of the target user in step 203. That is to say, it is more likely to screen out true positive cases and less likely to screen out false negative cases under less false negative scenario, which meets the actual needs of less false negatives.

Step 4034B, candidate disease suffering probability thresholds in the set of candidate disease suffering probability thresholds corresponding to the preset heart disease are ranked in an order of the corresponding specificity from high to low.

That is, if it is determined in step 4033 that the current application scenario is a less false positive scenario, in step 4034B, the candidate disease suffering probability thresholds ThPᵢ[q] in the set ThPᵢ of candidate disease suffering probability thresholds corresponding to the preset heart disease Dᵢ can be ranked in the order of the specificities SP_{i,j} corresponding to the candidate disease suffering probability thresholds ThPᵢ[q] from high to low to obtain a second ranking result of each candidate disease suffering probability threshold ThPᵢ[q] in the candidate disease suffering probability threshold set ThPᵢ.

Step 4035B may be performed after execution of step 4034B.

Step 4035B, a candidate disease suffering probability threshold ranked at a preset higher specificity ranking position in the set of candidate disease suffering probability thresholds corresponding to the preset heart disease is determined as the heart disease suffering probability threshold corresponding to the preset heart disease.

That is, the candidate disease suffering probability threshold ThPᵢ[q] ranked at the preset higher specificity ranking position in the set ThPᵢ of candidate disease suffering probability thresholds corresponding to the preset heart disease Dᵢ is determined as the heart disease suffering probability threshold corresponding to the preset heart disease Dᵢ. The preset higher specificity ranking position may be a position ranked in the first second preset proportion in the candidate disease suffering probability threshold set ThPᵢ, such as the last one ranked in first 5%. The preset higher specificity ranking position may also be a first second preset position ranked in the candidate disease suffering probability threshold set ThPᵢ, such as ranking in the fifth.

Here, the specificity SP_{i,j} corresponding to the candidate disease suffering probability threshold ThPᵢ[q] ranked at the preset higher specificity ranking position in the candidate disease suffering probability threshold set ThPᵢ is relatively high, the candidate disease suffering probability threshold ThPᵢ[q] corresponding to the relatively high specificity SP_{i,j} is determined as the heart disease suffering probability threshold corresponding to the preset heart disease Dᵢ, and a relatively high true negative rate and a relatively low false positive rate can be achieved in the process of generating the heart disease diagnosis result information of the target user in step 203. That is to say, it is more likely to screen out true negative cases and less likely to screen out false positive cases under less false positive scenario, which meets the actual needs of less false positive scenario.

Using the above-described alternative embodiment of the disease suffering probability threshold determination step 400, customization of corresponding disease suffering probability thresholds for different application scenarios can be achieved, which can meet the actual needs of different application scenarios.

In some alternative embodiments, the flow 200 of the above-described electrocardiogram analysis method may further include the following step 204:
step 204, the heart disease diagnosis result information of the target user is presented.

Here, the heart disease diagnosis result information of the target user may be presented on an information presentation device locally connected to the executive subject (e.g. a display device and/or a speaker locally connected to the executive subject).

Optionally, the heart disease diagnosis result information of the target user may be transmitted to other electronic devices connected to the executive subject through a network, and the heart disease diagnosis result information of the target user may be presented on an information presentation device locally connected to the other electronic devices.

In particular, the heart disease diagnosis result information of the target user may be presented on a display device, for example in the form of text or images. The voice corresponding to the heart disease diagnosis result information of the target user may also be played on a sound playing device. No specific limitations are made in the present disclosure.

According to the electrocardiogram analysis method provided by the above-described embodiments of the present disclosure, first at least one electrocardiogram data segment to be analyzed of the target user is acquired. Then, each electrocardiogram data segment to be analyzed is input into the pre-trained first electrocardiogram analysis model to obtain the heart disease suffering probability vector corresponding to each electrocardiogram data segment to be analyzed. Finally, the heart disease diagnosis result information of the target user is generated on the basis of the heart disease suffering probability vector corresponding to each electrocardiogram data segment to be analyzed. That is, the heart disease diagnosis result information of the target user is obtained by analyzing the electrocardiogram data of the target user.

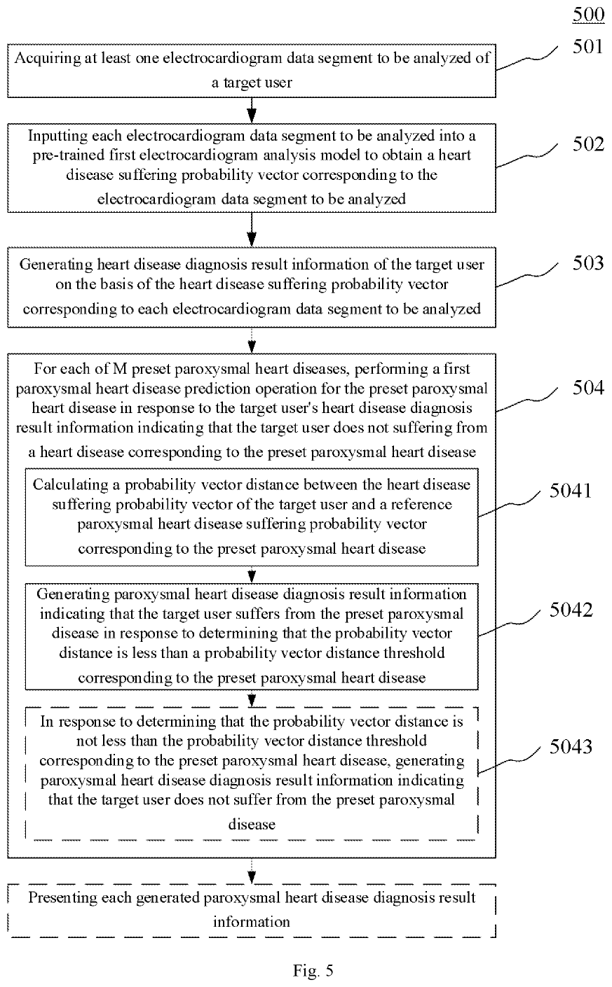

With continued reference to FIG. 5, a flow 500 of yet another embodiment of the electrocardiogram analysis method according to the present disclosure is shown. The electrocardiogram analysis method includes the following steps:
Step 501, at least one electrocardiogram data segment to be analyzed of the target user is acquired.
Step 502, each electrocardiogram data segment to be analyzed is input into a pre-trained first electrocardiogram analysis model to obtain a heart disease suffering probability vector corresponding to the electrocardiogram data segment to be analyzed.
Step 503, on the basis of the heart disease suffering probability vector corresponding to each electrocardiogram data segment to be analyzed, heart disease diagnosis result information of the target user is generated.

In this embodiment, the specific operations of step 501, step 502, and step 503 and the technical effects produced thereby are substantially the same as the operations and effects of step 201, step 202, and step 203 in the embodiment shown in FIG. 2A, which will not be described in detail herein.

Step 504, for each of M preset paroxysmal heart diseases, a first paroxysmal heart disease prediction operation is performed for the preset paroxysmal heart disease in response to the target user's heart disease diagnosis result information indicating that a probability of the target user suffering from a heart disease corresponding to the preset paroxysmal heart disease falls within a preset lower disease suffering probability range.

In practice, some heart diseases are paroxysmal, i.e. paroxysmal heart diseases. However, for patients with the paroxysmal heart disease, since most electrocardiogram data are usually collected in only a few tens of seconds, if patients do not show relevant symptoms when performing electrocardiogram examination, it is difficult for even experienced doctors to find problems from electrocardiogram data and make specific diagnosis of what kind of paroxysmal heart disease it is. However, the paroxysmal heart disease poses serious health risks. Therefore, the discovery of the paroxysmal heart disease is important.

In order to determine whether the target user has the paroxysmal heart disease, in this embodiment, an executive subject of the electrocardiogram analysis method (e.g. the client shown in FIG. 1) may perform the first paroxysmal heart disease prediction operation for the preset paroxysmal heart disease, for each of the M preset paroxysmal heart diseases, in response to the target user's heart disease diagnosis result information indicating that the probability of the target user suffering from a heart disease corresponding to the preset paroxysmal heart disease falls within the preset lower disease suffering probability range. Here, the preset lower disease suffering probability range may be a preset relatively lower disease suffering probability range. For example, the preset lower disease suffering probability range may be less than or equal to a lowest disease suffering probability threshold corresponding to a heart disease corresponding to the preset paroxysmal heart disease. The lowest disease suffering probability threshold corresponding to the heart disease corresponding to the preset paroxysmal heart disease may be less than or equal to the disease suffering probability threshold of the corresponding heart disease. It can also be understood that if the probability of the target user suffering from a heart disease corresponding to the preset paroxysmal heart disease is lower, it is further determined whether the target user suffers from the preset paroxysmal heart disease.

Here, M is a positive integer less than or equal to K, and the heart diseases corresponding to the M preset paroxysmal heart diseases belong to K preset heart diseases. Namely, each preset paroxysmal heart disease corresponds to a corresponding preset heart disease, and heart diseases corresponding to each preset paroxysmal heart disease in the M preset paroxysmal heart diseases belong to K preset heart diseases. However, there may be some preset heart diseases among the K preset heart diseases that do not correspond to the corresponding preset paroxysmal heart diseases.

In some alternative embodiments, the M preset paroxysmal heart diseases may be selected from the following paroxysmal heart diseases: paroxysmal sinus tachycardia, paroxysmal sinus bradycardia, paroxysmal premature atrial contraction, paroxysmal premature junctional contraction, paroxysmal premature ventricular contraction, paroxysmal supraventricular tachycardia, paroxysmal ventricular tachycardia, paroxysmal atrial flutter, paroxysmal atrial fibrillation, paroxysmal atrial escape, paroxysmal junctional escape, paroxysmal ventricular escape, paroxysmal sinus arrhythmia, paroxysmal sinus arrest and paroxysmal supraventricular premature beats. Optionally, M is a positive integer greater than or equal to 2.

It is assumed that that m is a positive integer from 1 to M and Pₘ is the mth preset paroxysmal heart disease of the M preset paroxysmal heart diseases. i is a positive integer of 1-K, and Dᵢ is the ith heart disease among K preset heart diseases. In step 504, for each preset paroxysmal heart disease Pₘ, if the heart disease diagnosis result information of the target user obtained in step 503 indicates that the probability of the target user suffer from a heart disease Dᵢ corresponding to the preset paroxysmal heart disease Pm belongs to a preset lower disease suffering probability range, the first paroxysmal heart disease prediction operation may be performed for the preset paroxysmal heart disease Pₘ.

Here, the first paroxysmal heart disease prediction operation may include the following steps 5041 and 5042:
Step 5041, a probability vector distance between the heart disease suffering probability vector of the target user and a reference paroxysmal heart disease suffering probability vector corresponding to the preset paroxysmal heart disease is calculated.

It should be noted that each preset paroxysmal heart disease Pₘ may correspond to a corresponding reference paroxysmal heart disease suffering probability vector Vₘ. Here, the reference paroxysmal heart disease suffering probability vector Vₘ corresponding to the preset paroxysmal heart disease Pₘ is used to characterize a reference probability of a patient diagnosed with the preset paroxysmal heart disease Pₘ suffering from each of the K preset heart diseases. Alternatively, the reference paroxysmal heart disease suffering probability vector Vₘ corresponding to the preset paroxysmal heart disease Pₘ can be understood as: inputting an electrocardiogram data segment of the patient diagnosed with the preset paroxysmal heart disease Pₘ into the first electrocardiogram analysis model to obtain a first heart disease suffering probability vector, and the distance between the above-mentioned first heart disease suffering probability vector and the reference paroxysmal heart disease suffering probability vector Vₘ should be less than a probability vector distance threshold corresponding to the preset paroxysmal heart disease. Conversely, an electrocardiogram data segment of the patient diagnosed as not suffering from the preset paroxysmal heart disease Pₘ is input into the first electrocardiogram analysis model to obtain a second heart disease suffering probability vector, and the distance between the above-mentioned second heart disease suffering probability vector and the reference paroxysmal heart disease suffering probability vector Vₘ should not be less than the probability vector distance threshold corresponding to the preset paroxysmal heart disease.

Here, various distance calculation methods may be used to calculate the probability vector distance between the heart disease suffering probability vector of the target user and the reference paroxysmal heart disease suffering probability vector corresponding to the preset paroxysmal heart disease. For example, the probability vector distance may be a distance between vectors such as a Educlidean metric, a Manhattan Distance, a Chebyshev Distance, a Mahalanobis Distance, etc. Alternatively, the probability vector distance may be various loss functions, such as relative entropy, also known as Kullback-Leibler divergence or information divergence. No specific limitations are made in the present disclosure.

Step 5042, in response to determining that the probability vector distance is less than a probability vector distance threshold corresponding to the preset paroxysmal heart disease, paroxysmal heart disease diagnosis result information indicating that the target user suffers from the preset paroxysmal disease is generated.

Here, it is assumed that the probability vector distance threshold corresponding to the preset paroxysmal heart disease Pₘ is ThDₘ. Therefore, if the probability vector distance calculated in step 5041 is less than the probability vector distance threshold ThDm corresponding to the preset paroxysmal heart disease Pₘ, it is indicated that the distance between the heart disease suffering probability vector of the target user and the heart disease suffering probability vector obtained by inputting the electrocardiogram data segment of the patient diagnosed with the preset paroxysmal heart disease Pₘ into the first electrocardiogram analysis model is small, the target user should also be determined to suffer from the preset paroxysmal heart disease Pₘ, and thus the paroxysmal heart disease diagnosis result information indicating that the target user suffers from the preset paroxysmal heart disease Pₘ can be generated.

In some alternative embodiments, the first paroxysmal heart disease prediction operation may further include the following step 5043:
Step 5043, in response to determining that the probability vector distance is not less than a probability vector distance threshold corresponding to the preset paroxysmal heart disease, paroxysmal heart disease diagnosis result information indicating that the target user does not suffer from the preset paroxysmal disease is generated.

That is, if the probability vector distance calculated in step 5041 is not less than the probability vector distance threshold ThDm corresponding to the preset paroxysmal heart disease Pₘ, it is indicated that the distance between the heart disease suffering probability vector of the target user and the heart disease suffering probability vector obtained by inputting the electrocardiogram data segment of the patient diagnosed with the preset paroxysmal heart disease Pₘ into the first electrocardiogram analysis model is large, the target user should be determined to not suffer from the preset paroxysmal heart disease Pₘ, and therefore, paroxysmal heart disease diagnosis result information indicating that the target user does not suffer from the preset paroxysmal heart disease Pₘ can be generated.

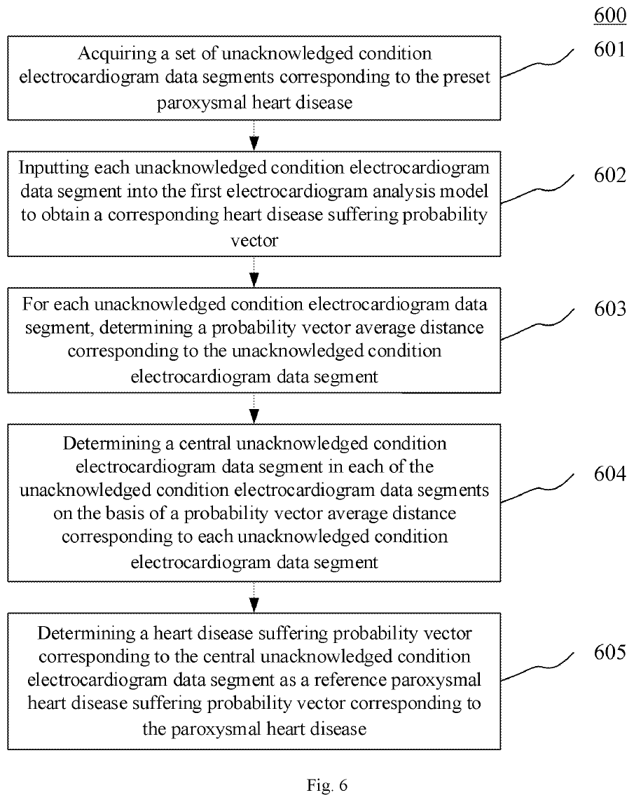

In some alternative embodiments, the reference paroxysmal heart disease suffering probability vector Vₘ corresponding to each preset paroxysmal heart disease Pₘ may be obtained by performing, for each preset paroxysmal heart disease Pₘ, a probability vector generation step 600 as shown in FIG. 6, and the probability vector generation step 600 includes the following steps 601 to 605:
Here, an executive subject of the probability vector generation step may be the same as or different from the executive subject of the electrocardiogram analysis method described above. If the executive subject of the probability vector generation step is the same as the executive subject of the above-mentioned electrocardiogram analysis method, the executive subject of the probability vector generation step may store the reference paroxysmal heart disease suffering probability vector Vₘ locally in the above-mentioned executive subject after obtaining the reference paroxysmal heart disease suffering probability vector Vₘ corresponding to the preset paroxysmal heart disease Pₘ. If the executive subject of the probability vector generation step is different from the executive subject of the above-mentioned electrocardiogram analysis method, the executive subject of the probability vector generation step may send the reference paroxysmal heart disease suffering probability vector Vₘ to the executive subject of the above-mentioned electrocardiogram analysis method after obtaining the reference paroxysmal heart disease suffering probability vector Vₘ.

Step 601, a set of unacknowledged condition electrocardiogram data segments corresponding to the preset paroxysmal heart disease is acquired.

Here, for each preset paroxysmal heart disease Pₘ, a set Sₘ of unacknowledged condition electrocardiogram data segments corresponding to the preset paroxysmal heart disease Pₘ may be acquired.

Here, each unacknowledged condition electrocardiogram data segment is an electrocardiogram data segment obtained after segmenting unacknowledged condition electrocardiogram data. While the unacknowledged condition electrocardiogram data is electrocardiogram data of the electrocardiogram examination for the subject diagnosed with the preset heart disease Di corresponding to the paroxysmal heart disease Pₘ, which is labeled as the corresponding subject does not suffer from the preset heart disease corresponding to the paroxysmal heart disease according to the unacknowledged condition electrocardiogram data segment.

In some alternative embodiments, the set Sₘ of unacknowledged condition electrocardiogram data segments corresponding to the preset paroxysmal heart disease Pₘ may be acquired by any one of the following implementations:
a first implementation: first, the first training data, the verification data and the test data of the subject diagnosed with the preset heart disease Dᵢ corresponding to the preset paroxysmal heart disease Pₘ are selected in the first training data set described in step 301, the verification data set described in step 302 or the test data set described in step 401. Then, first training data, verification data, and test data of the unacknowledged condition in which the labeled heart disease suffering probability vector is used for indicating not suffering from the preset heart disease Dᵢ corresponding to the preset paroxysmal heart disease Pₘ are selected in the selected first training data, verification data, and test data. Finally, the electrocardiogram data segments in the above-mentioned first training data, verification data and test data of the unacknowledged condition are acquired as the set Sₘ of the unacknowledged condition electrocardiogram data segments corresponding to the preset paroxysmal heart disease Pₘ.

A second implementation: first, historical electrocardiogram data (e.g. electrocardiogram data examined in a hospital or electrocardiogram data examined using a home portable electrocardiogram examination apparatus) of a subject diagnosed with the preset heart disease Di corresponding to the preset paroxysmal heart disease Pₘ is acquired. Then, unacknowledged condition electrocardiogram data labeled as not suffering from the preset heart disease Di corresponding to the preset paroxysmal heart disease Pₘ in the above-mentioned historical electrocardiogram data is acquired. Finally, the above-mentioned unacknowledged condition electrocardiogram data is segmented using the segmentation processing method described in the above-mentioned step 2012, so as to obtain the unacknowledged condition electrocardiogram data segment set.

A third implementation: first, Holter data of a subject diagnosed with the preset heart disease Di corresponding to the preset paroxysmal heart disease Pₘ is acquired. Then, unacknowledged condition Holter data labeled as not suffering from the preset heart disease Dᵢ corresponding to the preset paroxysmal heart disease Pₘ in the Holter data is acquired. Finally, the above-mentioned unacknowledged condition electrocardiogram data is segmented using the segmentation processing method described in the above-mentioned step 2012, so as to obtain the unacknowledged condition electrocardiogram data segment set.

Step 602, each unacknowledged condition electrocardiogram data segment is input into the first electrocardiogram analysis model to obtain a corresponding heart disease suffering probability vector.

Step 603, for each unacknowledged condition electrocardiogram data segment, a probability vector average distance corresponding to the unacknowledged condition electrocardiogram data segment is determined.

Here, the probability vector average distance corresponding to the unacknowledged condition electrocardiogram data segment is an average distance between a heart disease suffering probability vector corresponding to the unacknowledged condition electrocardiogram data segment and heart disease suffering probability vectors corresponding to other unacknowledged condition electrocardiogram data segments in the unacknowledged condition electrocardiogram data segment set except for the unacknowledged condition electrocardiogram data segment.

It is assumed that the set Sₘ of the unacknowledged condition electrocardiogram data segments corresponding to the preset paroxysmal heart disease Pₘ includes Q unacknowledged condition electrocardiogram data segments, q is a positive integer from 1 to Q, and Sₘ[q] is the qth unacknowledged condition electrocardiogram data segment in Sₘ. In step 603, for each unacknowledged condition electrocardiogram data segment Sₘ[q], the average value of the distances between the heart disease suffering probability vector corresponding to Sₘ[q] and the heart disease suffering probability vectors corresponding to other unacknowledged condition electrocardiogram data segments in the unacknowledged condition electrocardiogram data segment set Sₘ except for Sₘ[q] is calculated, namely, the probability vector average distance corresponding to the unacknowledged condition electrocardiogram data segment Sₘ[q].

Step 604, a central unacknowledged condition electrocardiogram data segment in each unacknowledged condition electrocardiogram data segment is determined on the basis of a probability vector average distance corresponding to each unacknowledged condition electrocardiogram data segment.

Here, the central unacknowledged condition electrocardiogram data segment is used to characterize the center of each unacknowledged condition electrocardiogram data segment.

Step 605, a heart disease suffering probability vector corresponding to the central unacknowledged condition electrocardiogram data segment is determined as a reference paroxysmal heart disease suffering probability vector corresponding to the paroxysmal heart disease.

Then, the reference paroxysmal heart disease suffering probability vector corresponding to the paroxysmal heart disease will be at the center of the heart disease suffering probability vector corresponding to each unacknowledged condition electrocardiogram data segment, and can be used to characterize the heart disease suffering probability vector corresponding to each unacknowledged condition electrocardiogram data segment that is diagnosed as suffering from the paroxysmal heart disease but labeled as not suffering from the heart disease corresponding to the paroxysmal heart disease according to the heart disease data segment.

Optionally, step 604 may be performed as follows: the corresponding unacknowledged condition electrocardiogram data segment with the smallest probability vector average distance in each unacknowledged condition electrocardiogram data segment is determined as the central unacknowledged condition electrocardiogram data segment. Thus, the average distance between the heart disease suffering probability vector corresponding to the central unacknowledged condition electrocardiogram data segment and the heart disease suffering probability vectors corresponding to other unacknowledged condition electrocardiogram data segments in the unacknowledged condition electrocardiogram data segment set is the smallest, and it can be considered that the heart disease suffering probability vector corresponding to the central unacknowledged condition electrocardiogram data segment is at the center of the heart disease suffering probability vector corresponding to each unacknowledged condition electrocardiogram data segment. Further, the reference paroxysmal heart disease suffering probability vector corresponding to the paroxysmal heart disease determined in step 605 will also be at the center of the heart disease suffering probability vector corresponding to each unacknowledged condition electrocardiogram data segment. The reference heart disease suffering probability vector for each preset paroxysmal heart disease determined in this alternative manner may improve the accuracy of generating paroxysmal heart disease diagnosis result information indicating that the target user suffers from the preset paroxysmal disease during the process of performing the first paroxysmal heart disease prediction operation in step 504.

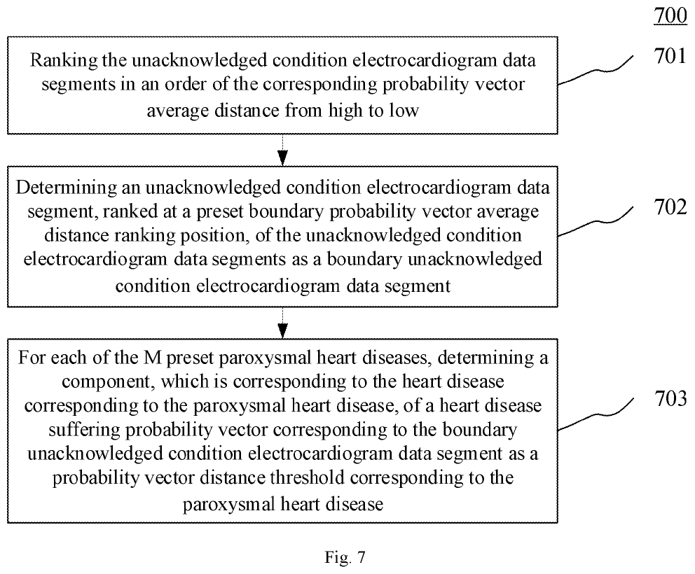

In some alternative embodiments, the probability vector distance threshold corresponding to each of the M preset paroxysmal heart diseases may be obtained by a probability vector distance threshold determination step 700 as shown in FIG. 7, the probability vector distance threshold determination step 700 including the following steps 701 to 703:
Step 701, the unacknowledged condition electrocardiogram data segments are ranked in an order of the corresponding probability vector average distance from high to low.
Step 702, an unacknowledged condition electrocardiogram data segment, ranked at a preset boundary probability vector average distance ranking position, of the unacknowledged condition electrocardiogram data segments is determined as a boundary unacknowledged condition electrocardiogram data segment.

Here, the preset boundary probability vector average distance ranking position may be a position ranked in the first third preset percentage in the unacknowledged condition electrocardiogram data segments, such as the last one ranked in first 5%. The preset boundary probability vector average distance ranking position may also be a first third preset position ranked in the unacknowledged condition electrocardiogram data segments, such as ranking in the second. Since the preset boundary probability vector average distance ranking position is a position ranked near the top in the unacknowledged condition electrocardiogram data segments, accordingly, the probability vector average distance corresponding to the boundary unacknowledged condition electrocardiogram data segment is also relatively large, and the heart disease suffering probability vector corresponding to the boundary unacknowledged condition electrocardiogram data segment can be used to characterize the boundary of the heart disease suffering probability vector corresponding to each unacknowledged condition electrocardiogram data segment.

Step 703, for each of the M preset paroxysmal heart diseases, a component, which is corresponding to the heart disease corresponding to the paroxysmal heart disease, of the heart disease suffering probability vector corresponding to the boundary unacknowledged condition electrocardiogram data segment is determined as a probability vector distance threshold corresponding to the paroxysmal heart disease.

The heart disease suffering probability vector corresponding to the boundary unacknowledged condition electrocardiogram data segment can be used to characterize the boundary of the heart disease suffering probability vector corresponding to each unacknowledged condition electrocardiogram data segment. A heart disease suffering probability vector within the boundary can be considered to characterize suffering from the paroxysmal heart disease, while a heart disease suffering probability vector outside the boundary can be considered to characterize not suffering from the paroxysmal heart disease. Thus, the component, which is corresponding to the heart disease corresponding to the paroxysmal heart disease, of the heart disease suffering probability vector corresponding to the boundary unacknowledged condition electrocardiogram data segment may be determined as the probability vector distance threshold corresponding to the paroxysmal heart disease.

In some alternative embodiments, the above-mentioned executive subject may also perform the following step 505 after performing step 504:
Step 505, the generated paroxysmal heart disease diagnosis result information is presented.

Here, each paroxysmal heart disease diagnosis result information generated in step 504 may be presented on an information presentation device locally connected to the executive subject (e.g. a display device and/or a speaker locally connected to the executive subject).

Optionally, each paroxysmal heart disease diagnosis result information generated in step 504 may be transmitted to other electronic devices connected to the executive subject through a network, and each paroxysmal heart disease diagnosis result information generated in step 504 may be presented on an information presentation device locally connected to the other electronic devices.

In particular, each paroxysmal heart disease diagnosis result information generated in step 504 may be presented on a display device. It may for example be presented in the form of text or images. Voice corresponding to each paroxysmal heart disease diagnosis result information generated in step 504 may also be played on a sound playing device. No specific limitations are made in the present disclosure. With this alternative embodiment, real-time analysis of the target user's electrocardiogram data and presentation of paroxysmal heart disease diagnosis result information can be achieved for timely reference by the target user or medical personnel.

As can be seen from FIG. 5, compared with the embodiment corresponding to FIG. 2A, the flow 500 of the electrocardiogram analysis method in this embodiment includes a step 504 of further confirming whether the target user diagnosed with the preset heart disease with a low disease suffering probability suffers from the corresponding preset paroxysmal heart disease. Thus, the solution described in this embodiment can further generate paroxysmal heart disease diagnosis result information indicating which preset paroxysmal heart disease the target user suffers from, further enriching the type of the diagnosis result information content for analyzing the electrocardiogram data.

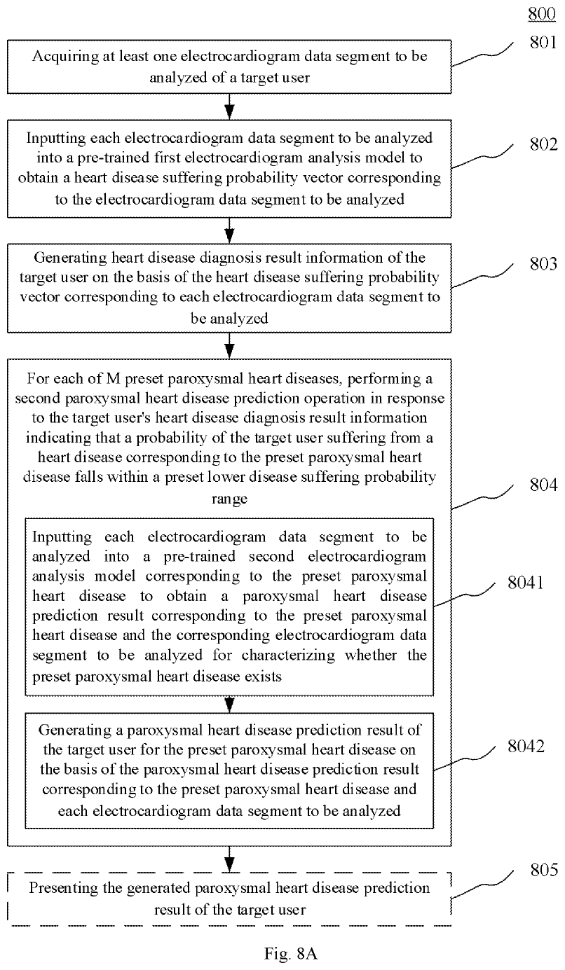

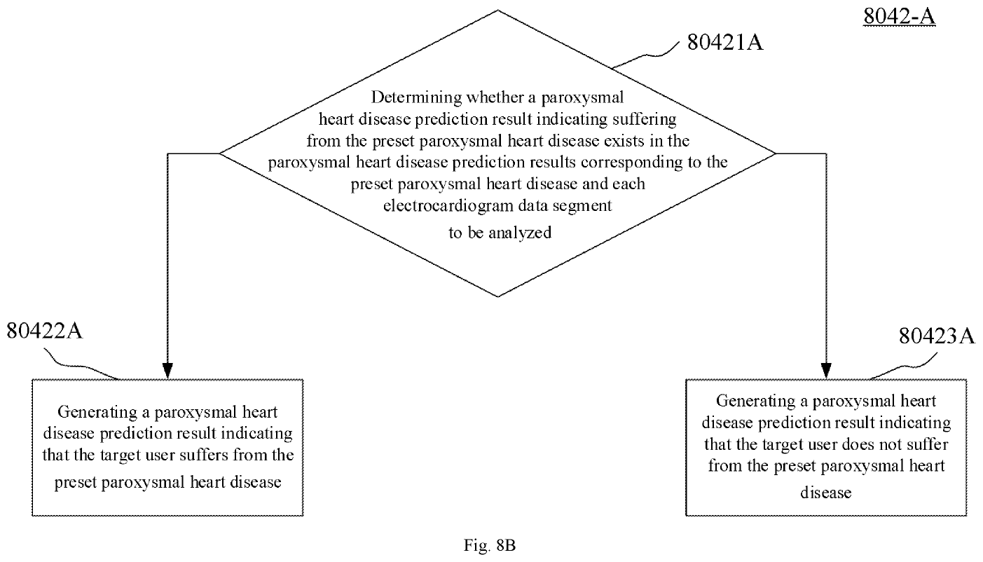

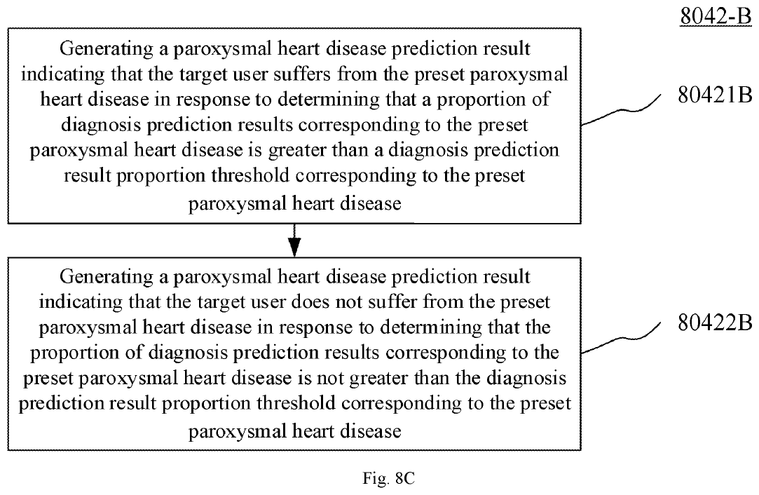

With continued reference to FIG. 8A, a flow 800 of another embodiment of the electrocardiogram analysis method according to the present disclosure is shown. The electrocardiogram analysis method includes the following steps:
Step 801, at least one electrocardiogram data segment to be analyzed of a target user is acquired.
Step 802, each electrocardiogram data segment to be analyzed is input into a pre-trained first electrocardiogram analysis model to obtain a heart disease suffering probability vector corresponding to the electrocardiogram data segment to be analyzed.
Step 803, on the basis of the heart disease suffering probability vector corresponding to each electrocardiogram data segment to be analyzed, heart disease diagnosis result information of the target user is generated.

In this embodiment, the specific operations of step 801, step 802, and step 803 and the technical effects produced thereby are substantially the same as the operations and effects of step 201, step 202, and step 203 in the embodiment shown in FIG. 2A, which will not be described in detail herein.

Step 804, for each of the M preset paroxysmal heart diseases, a second paroxysmal heart disease prediction operation is performed in response to the target user's heart disease diagnosis result information indicating that a probability of the target user suffering from a heart disease corresponding to the preset paroxysmal heart disease falls within a preset lower disease suffering probability range.

In order to determine whether the target user suffers from the paroxysmal heart disease, in this embodiment, an executive subject of the electrocardiogram analysis method (e.g. the client shown in FIG. 1) may perform the second paroxysmal heart disease prediction operation for each of the M preset paroxysmal heart diseases in response to the target user's heart disease diagnosis result information indicating that the probability of the target user suffering from the heart disease corresponding to the preset paroxysmal heart disease falls within the preset lower disease suffering probability range. M is a positive integer less than or equal to K, and heart diseases corresponding to M preset paroxysmal heart diseases belong to K preset heart diseases. It is assumed that m is a positive integer of 1-M, and Pₘ is the mth preset paroxysmal heart disease of the M preset paroxysmal heart diseases. i is a positive integer of 1-K, and Dᵢ is the ith heart disease among K preset heart diseases. In step 804, for each preset paroxysmal heart disease Pₘ, if the heart disease diagnosis result information of the target user obtained in step 803 indicates that the probability of the target user suffering from the heart disease Di corresponding to the preset paroxysmal heart disease Pₘ falls within the preset lower disease suffering probability range, the second paroxysmal heart disease prediction operation is performed for the preset paroxysmal heart disease Pₘ.

Here, the second paroxysmal heart disease prediction operation may include the following steps 8041 to 8042:
Step 8041, each electrocardiogram data segment to be analyzed is input into a pre-trained second electrocardiogram analysis model corresponding to the preset paroxysmal heart disease, so as to obtain a paroxysmal heart disease prediction result corresponding to the preset paroxysmal heart disease and the corresponding electrocardiogram data segment to be analyzed, which is used for characterizing whether the preset paroxysmal heart disease exists.

It is assumed that J electrocardiogram data segments to be analyzed are acquired in step 801, j is a positive integer from 1 to J, and SEⱼ is the jth electrocardiogram data segment to be analyzed in the J electrocardiogram data segments to be analyzed.

Step 8041 refers to inputting electrocardiogram data segments SE₁, SE₂, . . . SE_{J} to be analyzed into a second electrocardiogram analysis model corresponding to the preset paroxysmal heart disease Pₘ, respectively to respectively obtain paroxysmal heart disease prediction results R_{1,m}, R_{2,m}, . . . R_{J,m} corresponding to the electrocardiogram data segments SE₁, SE₂, . . . SE_{J} to be analyzed and used for characterizing whether the preset paroxysmal heart disease Pₘ exists.

Here, the second electrocardiogram analysis model corresponding to the preset paroxysmal heart disease Pₘ is used to characterize a correspondence between the electrocardiogram data segment and the paroxysmal heart disease prediction result for characterizing whether the preset paroxysmal heart disease Pₘ exists.

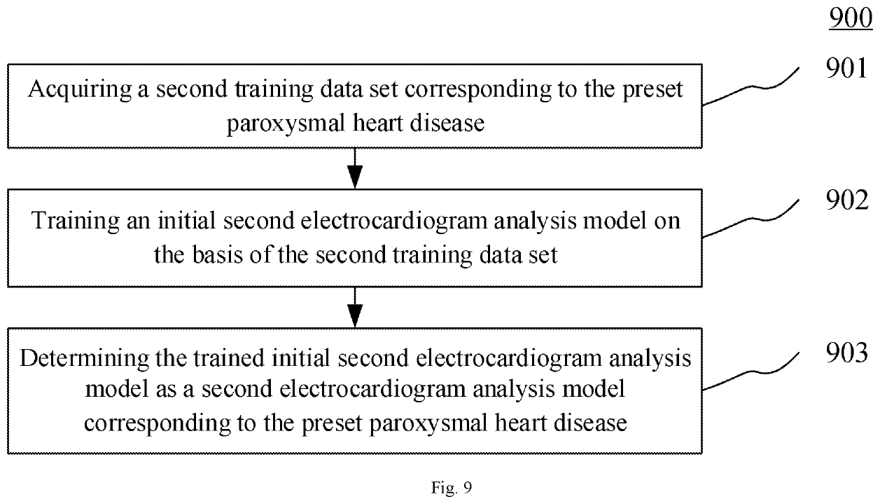

Here, the second electrocardiogram analysis model corresponding to the preset paroxysmal heart disease Pₘ may be pre-trained through the second training step 900 as shown in FIG. 9, and the second training step 900 may include the following steps 901 to 903:
Step 901, a second training data set corresponding to the preset paroxysmal heart disease is acquired.

Here, second training data in the second training data set corresponding to the preset paroxysmal heart disease Pₘ may include a sample electrocardiogram data segment and a corresponding labeled paroxysmal heart disease prediction result for characterizing whether the preset paroxysmal heart disease Pₘ is present.

When the sample electrocardiogram data segment is an unacknowledged condition electrocardiogram data segment corresponding to the preset paroxysmal heart disease Pₘ, the corresponding labeled paroxysmal heart disease prediction result is used to characterize suffering from the preset paroxysmal heart disease Pₘ. Here, the unacknowledged condition electrocardiogram data segment corresponding to the preset paroxysmal heart disease Pₘ is an electrocardiogram data segment, labeled as the corresponding subject according to the unacknowledged condition electrocardiogram data segment does not suffer from the preset heart disease Dᵢ corresponding to the paroxysmal heart disease Pₘ, in electrocardiogram data of electrocardiogram examination on a subject diagnosed as suffering from the preset heart disease Dᵢ corresponding to the paroxysmal heart disease Pₘ.

When the sample electrocardiogram data segment is a normal electrocardiogram data segment corresponding to the preset paroxysmal heart disease Pₘ, the corresponding labeled paroxysmal heart disease prediction result is used to characterize that the preset paroxysmal heart disease Pm does not exist. Here, the normal electrocardiogram data segment corresponding to the preset paroxysmal heart disease Pₘ is an electrocardiogram data segment in electrocardiogram data of electrocardiogram examination on a subject diagnosed as not suffering from the preset heart disease Dᵢ corresponding to the paroxysmal heart disease Pₘ.

Step 902, an initial second electrocardiogram analysis model is trained on the basis of the second training data set.

Here, the initial second electrocardiogram analysis model may be trained on the basis of the second training data set by using various machine learning methods.

Here, the initial second electrocardiogram analysis model may be various binary classification models. For example, the initial second electrocardiogram analysis model may be an artificial neural network (ANN), a deep learning (DL) model, a support vector machine (SVM), a random forest (RF), a decision tree (DT), a linear regression (LR), a logistic regression (LR) or the like. Step 903, the trained initial second electrocardiogram analysis model is determined as a second electrocardiogram analysis model corresponding to the preset paroxysmal heart disease.

Step 8042, on the basis of the paroxysmal heart disease prediction result corresponding to the preset paroxysmal heart disease and each electrocardiogram data segment to be analyzed, a paroxysmal heart disease prediction result of the target user is generated for the preset paroxysmal heart disease.

Here, the above-mentioned executive subject may generate the paroxysmal heart disease prediction result of the target user for the preset paroxysmal heart disease Pₘ on basis of the paroxysmal heart disease prediction results R_{1,m}, R_{2,m}, . . . R_{J,m} corresponding to the preset paroxysmal heart disease Pₘ and the electrocardiogram data segments SE₁, SE₂, . . . SE_{J} to be analyzed obtained in step 8041 in various manners according to the needs of specific application scenarios.

Here, the paroxysmal heart disease prediction result of the target user generated for the preset paroxysmal heart disease Pₘ may be in various forms. For example, text, an image, and voice data may be included, but are not limited thereto.

Here, the paroxysmal heart disease prediction result of the target user generated for the preset paroxysmal heart disease Pₘ may be used to indicate that the target user is diagnosed to suffer from the preset paroxysmal heart disease Pₘ, or to indicate that the target user is not diagnosed to suffer from the preset paroxysmal heart disease Pₘ, or may also be used to indicate the degree to which the target user suffers from the preset paroxysmal heart disease Pₘ. Here, the degree information may be represented by numerical values or by words. For example, the degree information may be a degree value of 0-1. The degree information may also include, for example, “the risk of suffering from the preset paroxysmal heart disease Pₘ is very high”, “the risk of suffering from the preset paroxysmal heart disease Pₘ is high”, “the risk of suffering from the preset paroxysmal heart disease Pₘ is relatively low”, “the risk of suffering from the preset paroxysmal heart disease Pₘ is low”, etc.

In some alternative embodiments, step 8042 may include steps 80421A and 80422A as shown in FIG. 8B:
Step 80421A, whether a paroxysmal heart disease prediction result indicating suffering from the preset paroxysmal heart disease exists in the paroxysmal heart disease prediction results corresponding to the preset paroxysmal heart disease and each electrocardiogram data segment to be analyzed is determined.

If it is determined that the paroxysmal heart disease prediction result indicating suffering from the preset paroxysmal heart disease exists, it is indicated that at least one electrocardiogram data segment to be analyzed among the electrocardiogram data segments to be analyzed collects the moment when the target user has the preset paroxysmal heart disease Pₘ, indicating that the target user suffers from the preset paroxysmal heart disease Pₘ, and step 80422A may be performed.

Step 80422A, a paroxysmal heart disease prediction result indicating that the target user suffers from the preset paroxysmal heart disease is generated.

Optionally, in step 80421A, in the case where it is determined that the paroxysmal heart disease prediction result indicating suffering from the preset paroxysmal heart disease does not exist, it is indicated that no electrocardiogram data segment to be analyzed in the electrocardiogram data segments to be analyzed collects the moment when the target user has the preset paroxysmal heart disease Pₘ, indicating that it cannot be determined that the target user suffers from the preset paroxysmal heart disease Pₘ according to each electrocardiogram data segment to be analyzed, and step 80423A may be performed.

Step 80423A, a paroxysmal heart disease prediction result indicating that the target user does not suffer from the preset paroxysmal heart disease is generated.

In some alternative embodiments, step 8042 may include steps 80421B and 80422B as shown in FIG. 8C:
Step 80421B, in response to determining that a proportion of diagnosis prediction results corresponding to the preset paroxysmal heart disease is greater than a diagnosis prediction result proportion threshold corresponding to the preset paroxysmal heart disease, a paroxysmal heart disease prediction result indicating that the target user suffers from the preset paroxysmal heart disease is generated.

Here, the proportion of diagnosis prediction results corresponding to the preset paroxysmal heart disease Pₘ is a proportion of the number of the diagnosis prediction results corresponding to the preset paroxysmal heart disease divided by the total number (i.e. J) of the electrocardiogram data segments to be analyzed, and the number of the diagnosis prediction results corresponding to the preset paroxysmal heart disease is the number of paroxysmal heart disease prediction results indicating suffering from the preset paroxysmal heart disease in the paroxysmal heart disease prediction results corresponding to the preset paroxysmal heart disease and the electrocardiogram data segments to be analyzed.

In this alternative way, the paroxysmal heart disease prediction result for determining that the target user suffers from the preset paroxysmal heart disease Pₘ can be generated in the case that it can be determined that the target user suffers from the preset paroxysmal heart disease Pm according to the electrocardiogram data segments to be analyzed which exceed the diagnosis prediction result proportion threshold corresponding to the preset paroxysmal heart disease in the electrocardiogram data segments to be analyzed.

Optionally, step 8042 may also include step 80422B after step 80421B.

Step 80422B, in response to determining that the proportion of diagnosis prediction results corresponding to the preset paroxysmal heart disease is not greater than the diagnosis prediction result proportion threshold corresponding to the preset paroxysmal heart disease, a paroxysmal heart disease prediction result indicating that the target user does not suffer from the preset paroxysmal heart disease is generated.

In this alternative way, the paroxysmal heart disease prediction result that determines that the target user does not suffer from the preset paroxysmal heart disease Pₘ can be generated in the case that it cannot be determined that the target user suffers from the preset paroxysmal heart disease Pₘ according to the electrocardiogram data segments to be analyzed that exceed the diagnosis prediction result proportion threshold corresponding to the preset paroxysmal heart disease in the electrocardiogram data segments to be analyzed.

In some alternative embodiments, the above-mentioned executive subject may also perform the following step 805 after performing step 804:
Step 805, the generated paroxysmal heart disease prediction result of the target user is presented. Here, the paroxysmal heart disease prediction results of the target user generated for different preset paroxysmal heart diseases in step 804 may be presented on an information presentation device locally connected to the executive subject (e.g. a display device and/or a speaker locally connected to the executive subject).

Optionally, the paroxysmal heart disease prediction results of the target user generated for different preset paroxysmal heart diseases may be transmitted to other electronic devices connected to the executive subject through a network, and the paroxysmal heart disease prediction results of the target user generated for different preset paroxysmal heart diseases may be presented on an information presentation device locally connected to the other electronic devices.

In particular, the paroxysmal heart disease prediction results of the target user generated for different preset paroxysmal heart diseases may be presented on a display device. It may for example be presented in the form of text or images. Voice corresponding to the paroxysmal heart disease prediction results of the target user generated for different preset paroxysmal heart diseases may also be played on a sound playing device. No specific limitations are made in the present disclosure.

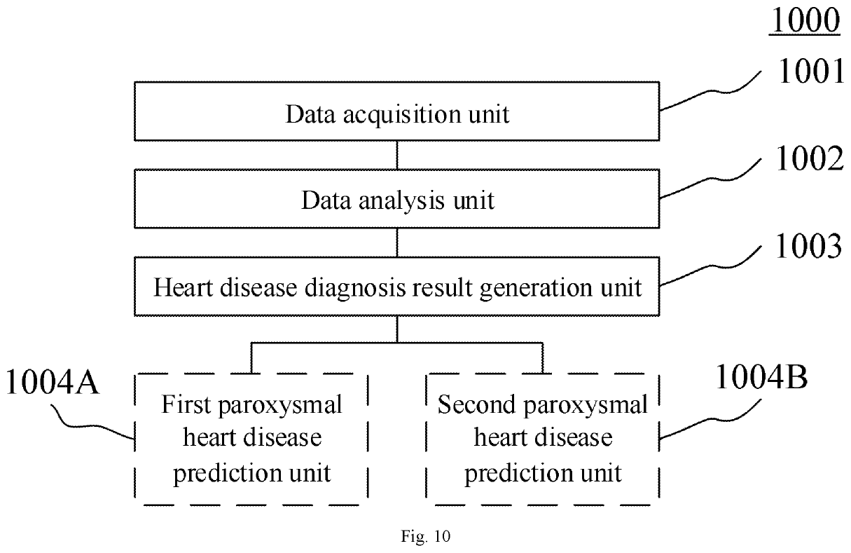

With further reference to FIG. 10, as an implementation of the method shown in the above figures, the present disclosure provides one embodiment of an electrocardiogram analysis apparatus corresponding to the method embodiment shown in FIG. 2A, and the apparatus may be particularly applied to various electronic devices.

As shown in FIG. 10, the electrocardiogram analysis apparatus 1000 in this embodiment includes: a data acquisition unit 1001, a data analysis unit 1002, and a heart disease diagnosis result generation unit 1003. The data acquisition unit 1001 is configured to acquire at least one electrocardiogram data segment to be analyzed of a target user; the data analysis unit 1002 is configured to input each electrocardiogram data segment to be analyzed into a pre-trained first electrocardiogram analysis model to obtain a heart disease suffering probability vector corresponding to the electrocardiogram data segment to be analyzed, wherein the heart disease suffering probability vector is used for characterizing a probability of suffering from each of K preset heart diseases, the first electrocardiogram analysis model is used for characterizing a correspondence between the electrocardiogram data segment and the heart disease suffering probability vector, and K is a positive integer; and the heart disease diagnosis result generation unit 1003 is configured to generate heart disease diagnosis result information of the target user on the basis of the heart disease suffering probability vector corresponding to each electrocardiogram data segment to be analyzed.

In this embodiment, in the electrocardiogram analysis apparatus 1000, the specific processes of the data acquisition unit 1001, the data analysis unit 1002, and the heart disease diagnosis result generation unit 1003 and the technical effects thereof may respectively refer to the relevant descriptions of steps 201, 202, and 203 in the corresponding embodiment of FIG. 2A, which will not be described in detail herein.

In some alternative embodiments, the first electrocardiogram analysis model may be pre-trained by the following first training steps: acquiring a first training data set, wherein first training data includes a sample electrocardiogram data segment and a corresponding labeled heart disease suffering probability vector, and the labeled heart disease suffering probability vector in the first training data is used for indicating a probability of a person, whom the sample electrocardiogram data segment in the first training data corresponds to and on whom collection is performed, suffering from each preset heart disease; training an initial first electrocardiogram analysis model on the basis of the first training data set; and determining the initial first electrocardiogram analysis model trained as the pre-trained first electrocardiogram analysis model.

In some alternative embodiments, the heart disease diagnosis result generation unit 1003 may be further configured to: for each preset heart disease, perform the following first diagnosis result information generating operations: determining a heart disease suffering probability of the target user suffering from the preset heart disease according to a component corresponding to the preset heart disease in the heart disease suffering probability vector corresponding to each electrocardiogram data segment to be analyzed; determining heart disease diagnosis result information corresponding to the heart disease suffering probability of the target user suffering from the preset heart disease according to a correspondence between a disease suffering probability range and heart disease diagnosis result information corresponding to the preset heart disease; and generating heart disease diagnosis result information of the target user suffering from the preset heart disease using the determined heart disease diagnosis result information. In some alternative embodiments, the determining a heart disease suffering probability of the target user suffering from the preset heart disease according to a component corresponding to the preset heart disease in the heart disease suffering probability vector corresponding to each electrocardiogram data segment to be analyzed may include: determining a mean value of components corresponding to the preset heart disease in the heart disease suffering probability vector corresponding to each electrocardiogram data segment to be analyzed as the heart disease suffering probability of the target user suffering from the preset heart disease; or ranking components corresponding to the preset heart disease in the heart disease suffering probability vector corresponding to each electrocardiogram data segment to be analyzed in an order from low to high, and determining a component ranked in a preset quantile as the heart disease suffering probability of the target user suffering from the preset heart disease. In some alternative embodiments, the determining a heart disease suffering probability of the target user suffering from the preset heart disease according to a component corresponding to the preset heart disease in the heart disease suffering probability vector corresponding to each electrocardiogram data segment to be analyzed may include: ranking components corresponding to the preset heart disease in the heart disease suffering probability vector corresponding to each electrocardiogram data segment to be analyzed in an order from low to high; in response to determining that the current application scenario is a less false positive scenario, determining a minimum value of the components corresponding to the preset heart disease in the heart disease suffering probability vector corresponding to each electrocardiogram data segment to be analyzed or a component ranked in a preset lower probability quantile as the heart disease suffering probability of the target user suffering from the preset heart disease; and in response to determining that the current application scenario is a less false negative scenario, determining a maximum value of the components corresponding to the preset heart disease in the heart disease suffering probability vector corresponding to each electrocardiogram data segment to be analyzed or a component ranked in a preset higher probability quantile as the heart disease suffering probability of the target user suffering from the preset heart disease.

In some alternative embodiments, the correspondence between a disease suffering probability range and heart disease diagnosis result information corresponding to the preset heart disease may include at least one of: a first correspondence for characterizing that a first disease suffering probability range corresponds to first diagnosis result information indicating that the preset heart disease is not diagnosed, wherein the first disease suffering probability range is less than a disease suffering probability threshold corresponding to the preset heart disease; and a second correspondence for characterizing that a second disease suffering probability range corresponds to second diagnosis result information indicating that the preset heart disease is diagnosed, wherein the second disease suffering probability range is greater than or equal to a disease suffering probability threshold corresponding to the preset heart disease.

In some alternative embodiments, the disease suffering probability threshold corresponding to each preset heart disease may be obtained by the following disease suffering probability threshold determination steps: acquiring a test data set, wherein test data includes a sample electrocardiogram data segment and a labeled heart disease suffering probability vector, and the labeled heart disease suffering probability vector in the test data is used for indicating a probability of a person, whom the sample electrocardiogram data segment in the test data corresponds to and on whom collection is performed, suffering from each preset heart disease; inputting sample electrocardiogram data segments in each of the test data into the first electrocardiogram analysis model to obtain a heart disease suffering probability vector test result corresponding to the test data; and for each preset heart disease, performing the following disease suffering probability threshold determination operations: acquiring a set of candidate disease suffering probability thresholds corresponding to the preset heart disease; for each candidate disease suffering probability threshold obtained, performing the following statistical operations: according to whether a vector component corresponding to the preset heart disease in the heart disease suffering probability vector test result corresponding to each of the test data is greater than the candidate disease suffering probability threshold, and whether a vector component corresponding to the preset heart disease in a labeled heart disease suffering probability vector in the corresponding test data is greater than the candidate disease suffering probability threshold, counting a sensitivity and specificity corresponding to the preset heart disease and the candidate disease suffering probability threshold; in response to determining that the current application scenario is a less false negative scenario, ranking the candidate disease suffering probability thresholds in the set of candidate disease suffering probability thresholds corresponding to the preset heart disease in an order of the corresponding sensitivity from high to low; determining a candidate disease suffering probability threshold ranked at a preset higher sensitivity ranking position in the set of candidate disease suffering probability thresholds corresponding to the preset heart disease as a heart disease suffering probability threshold corresponding to the preset heart disease; in response to determining that the current application scenario is a less false positive scenario, ranking the candidate disease suffering probability thresholds in the set of candidate disease suffering probability thresholds corresponding to the preset heart disease in an order of the corresponding specificity from high to low; and determining a candidate disease suffering probability threshold ranked at a preset higher specificity ranking position in the set of candidate disease suffering probability thresholds corresponding to the preset heart disease as a heart disease suffering probability threshold corresponding to the preset heart disease.

In some alternative embodiments, the heart disease diagnosis result generation unit 1003 may be further configured to: for each preset heart disease, perform the following second diagnosis result information generating operations: acquiring a set of disease suffering probability ranges corresponding to the preset heart disease; for each acquired disease suffering probability range, determining a proportion of data segments corresponding to the disease suffering probability range, wherein the proportion of data segments corresponding to the disease suffering probability range is a proportion of the number of components of the heart disease suffering probability vector belonging to the disease suffering probability range in the components corresponding to the preset heart disease in the heart disease suffering probability vector corresponding to each electrocardiogram data segment to be analyzed divided by the number of the electrocardiogram data segments to be analyzed; according to a correspondence between a disease suffering probability range and heart disease diagnosis result information corresponding to the preset heart disease, determining heart disease diagnosis result information corresponding to the disease suffering probability range with the largest proportion of the corresponding data segments; and generating heart disease diagnosis result information of the target user suffering from the preset heart disease using the determined heart disease diagnosis result information.

In some alternative embodiments, the heart disease diagnosis result generation unit 1003 may be further configured to: for each preset heart disease, in response to determining that a proportion of diagnosis electrocardiogram data segments corresponding to the preset heart disease is not less than a diagnosis proportion threshold corresponding to the preset heart disease, label the preset heart disease as a diagnosed heart disease, wherein the proportion of diagnosis electrocardiogram data segments corresponding to the preset heart disease is a proportion of the number of diagnosis electrocardiogram data segments corresponding to the preset heart disease divided by the total number of the electrocardiogram data segments to be analyzed, and the number of diagnosis electrocardiogram data segments corresponding to the preset heart disease is the number of components corresponding to the preset heart disease in a heart disease suffering probability vector corresponding to each electrocardiogram data segment to be analyzed, which are greater than a disease suffering probability threshold corresponding to the preset heart disease; and generate heart disease diagnosis result information indicating that the target user is diagnosed with a diagnosed heart disease among the preset heart diseases, which is labeled as a diagnosed heart disease.

In some alternative embodiments, the apparatus 1000 may further include a first paroxysmal heart disease prediction unit 1004A configured to: for each of M preset paroxysmal heart diseases, in response to the target user's heart disease diagnosis result information indicating that a probability of the target user suffering from a heart disease corresponding to the preset paroxysmal heart disease falls within a preset lower disease suffering probability range, perform the following first paroxysmal heart disease prediction operation for the preset paroxysmal heart disease: calculating a probability vector distance between a heart disease suffering probability vector of the target user and a reference paroxysmal heart disease suffering probability vector corresponding to the preset paroxysmal heart disease; and generating paroxysmal heart disease diagnosis result information for indicating that the target user suffers from the preset paroxysmal disease in response to determining that the probability vector distance is less than a probability vector distance threshold corresponding to the preset paroxysmal heart disease, wherein M is a positive integer less than or equal to the K, and heart diseases corresponding to the M preset paroxysmal heart diseases belong to the K preset heart diseases.

In some alternative embodiments, the first paroxysmal heart disease prediction operation may further include: in response to determining that the probability vector distance is not less than the probability vector distance threshold corresponding to the preset paroxysmal heart disease, generating paroxysmal heart disease diagnosis result information indicating that the target user does not suffer from the preset paroxysmal disease.

In some alternative embodiments, the reference paroxysmal heart disease suffering probability vector corresponding to each preset paroxysmal heart disease may be obtained by performing, for each preset paroxysmal heart disease, the following probability vector generation steps: acquiring a set of unacknowledged condition electrocardiogram data segments corresponding to the preset paroxysmal heart disease, wherein each of the unacknowledged condition electrocardiogram data segments is an electrocardiogram data segment obtained after segmenting unacknowledged condition electrocardiogram data, and the unacknowledged condition electrocardiogram data is electrocardiogram data of electrocardiogram examination on a subject diagnosed with a heart disease corresponding to the paroxysmal heart disease, which is labeled as the subject corresponding to the unacknowledged condition electrocardiogram data does not suffer from the heart disease corresponding to the paroxysmal heart disease; inputting each unacknowledged condition electrocardiogram data segment into the first electrocardiogram analysis model to obtain a corresponding heart disease suffering probability vector; for each of the unacknowledged condition electrocardiogram data segments, determining a probability vector average distance corresponding to the unacknowledged condition electrocardiogram data segment, wherein the probability vector average distance corresponding to the unacknowledged condition electrocardiogram data segment is an average distance between a heart disease suffering probability vector corresponding to the unacknowledged condition electrocardiogram data segment and heart disease suffering probability vectors corresponding to other unacknowledged condition electrocardiogram data segments in the unacknowledged condition electrocardiogram data segment set except for the unacknowledged condition electrocardiogram data segment; determining a central unacknowledged condition electrocardiogram data segment in each of the unacknowledged condition electrocardiogram data segments on the basis of a probability vector average distance corresponding to each of the unacknowledged condition electrocardiogram data segments; and determining a heart disease suffering probability vector corresponding to the central unacknowledged condition electrocardiogram data segment as a reference paroxysmal heart disease suffering probability vector corresponding to the paroxysmal heart disease.

In some alternative embodiments, the probability vector distance threshold corresponding to each of the M preset paroxysmal heart diseases may be obtained by: ranking the unacknowledged condition electrocardiogram data segments in an order of the corresponding probability vector average distance from high to low; determining an unacknowledged condition electrocardiogram data segment, ranked at a preset boundary probability vector average distance ranking position, of the unacknowledged condition electrocardiogram data segments as a boundary unacknowledged condition electrocardiogram data segment; and for each of the M preset paroxysmal heart diseases, determining a component , which is corresponding to the heart disease corresponding to the paroxysmal heart disease, of a heart disease suffering probability vector corresponding to the boundary unacknowledged condition electrocardiogram data segment as a probability vector distance threshold corresponding to the paroxysmal heart disease.

In some alternative embodiments, the apparatus 1000 may further include: a second paroxysmal heart disease prediction unit 1004B configured to: for each of M preset paroxysmal heart diseases, in response to the target user's heart disease diagnosis result information indicating that a probability of the target user suffering from a heart disease corresponding to the preset paroxysmal heart disease falls within a preset lower disease suffering probability range, perform the following second paroxysmal heart disease prediction operations: inputting each electrocardiogram data segment to be analyzed into a pre-trained second electrocardiogram analysis model corresponding to the preset paroxysmal heart disease to obtain a paroxysmal heart disease prediction result corresponding to the preset paroxysmal heart disease and the electrocardiogram data segment to be analyzed for characterizing whether the preset paroxysmal heart disease exists, wherein the second electrocardiogram analysis model corresponding to the preset paroxysmal heart disease is used for characterizing a correspondence between the electrocardiogram data segment and the paroxysmal heart disease prediction result; and generating a paroxysmal heart disease prediction result of the target user for the preset paroxysmal heart disease on the basis of the paroxysmal heart disease prediction result corresponding to the preset paroxysmal heart disease and each electrocardiogram data segment to be analyzed, wherein M is a positive integer less than or equal to K, and heart diseases corresponding to the M preset paroxysmal heart diseases belong to the K preset heart diseases. In some alternative embodiments, the generating a paroxysmal heart disease prediction result of the target user for the preset paroxysmal heart disease on the basis of the paroxysmal heart disease prediction result corresponding to the preset paroxysmal heart disease and each electrocardiogram data segment to be analyzed may include: determining whether a paroxysmal heart disease prediction result indicating suffering from the preset paroxysmal heart disease exists in the paroxysmal heart disease prediction results corresponding to the preset paroxysmal heart disease and each electrocardiogram data segment to be analyzed; and in response to determining presence, generating a paroxysmal heart disease prediction result indicating that the target user suffers from the preset paroxysmal heart disease.

In some alternative embodiments, the generating a paroxysmal heart disease prediction result of the target user for the preset paroxysmal heart disease on the basis of the paroxysmal heart disease prediction result corresponding to the preset paroxysmal heart disease and each electrocardiogram data segment to be analyzed may further include: in response to determining absence, generating a paroxysmal heart disease prediction result indicating that the target user does not suffer from the preset paroxysmal heart disease.

In some alternative embodiments, the generating a paroxysmal heart disease prediction result of the target user for the preset paroxysmal heart disease on the basis of the paroxysmal heart disease prediction result corresponding to the preset paroxysmal heart disease and each electrocardiogram data segment to be analyzed may include: generating a paroxysmal heart disease prediction result indicating that the target user suffers from the preset paroxysmal heart disease in response to determining that a proportion of diagnosis prediction results corresponding to the preset paroxysmal heart disease is greater than a diagnosis prediction result proportion threshold corresponding to the preset paroxysmal heart disease, wherein the proportion of the diagnosis prediction results corresponding to the preset paroxysmal heart disease is a proportion of the number of the diagnosis prediction results corresponding to the preset paroxysmal heart disease divided by the total number of the electrocardiogram data segments to be analyzed, and the number of the diagnosis prediction results corresponding to the preset paroxysmal heart disease is the number of paroxysmal heart disease prediction results indicating suffering from the preset paroxysmal heart disease in the paroxysmal heart disease prediction results corresponding to the preset paroxysmal heart disease and the electrocardiogram data segments to be analyzed.

In some alternative embodiments, the generating a paroxysmal heart disease prediction result of the target user for the preset paroxysmal heart disease on the basis of the paroxysmal heart disease prediction result corresponding to the preset paroxysmal heart disease and each electrocardiogram data segment to be analyzed may further include: generating a paroxysmal heart disease prediction result indicating that the target user does not suffer from the preset paroxysmal heart disease in response to determining that the proportion of diagnosis prediction results corresponding to the preset paroxysmal heart disease is not greater than the diagnosis prediction result proportion threshold corresponding to the preset paroxysmal heart disease. In some alternative embodiments, the data acquisition unit 1001 may be further configured to: acquire electrocardiogram data to be analyzed of a target user; and segment the electrocardiogram data to be analyzed to obtain at least one electrocardiogram data segment to be analyzed.

In some alternative embodiments, the data acquisition unit 1001 may be further configured to: resample the electrocardiogram data to be analyzed before the electrocardiogram data to be analyzed is segmented to obtain at least one electrocardiogram data segment to be analyzed, so that a sampling frequency of the electrocardiogram data to be analyzed is a preset sampling frequency.

In some alternative embodiments, the segmenting the electrocardiogram data to be analyzed to obtain at least one electrocardiogram data segment to be analyzed may include: performing average segmentation processing on the electrocardiogram data to be analyzed to obtain at least one electrocardiogram data segment to be analyzed, wherein each electrocardiogram data segment to be analyzed includes electrocardiogram data of F frames, and F is a positive integer. In some alternative embodiments, the K preset heart diseases may be K heart diseases selected from a preset heart disease set including: sinus tachycardia, sinus bradycardia, premature atrial contraction, premature junctional contraction, premature ventricular contraction, supraventricular tachycardia, ventricular tachycardia, atrial flutter, atrial fibrillation, atrial escape, junctional escape, ventricular escape, right bundle branch block, sinus arrhythmia, sinus arrest, supraventricular premature beats, paired supraventricular premature beats, bigeminy coupled rhythm of supraventricular premature beats, trigeminy of supraventricular premature beats, ventricular premature beats, paired ventricular premature beats, bigeminy coupled rhythm of ventricular premature beats, trigeminy of ventricular premature beats, supraventricular escape beats, pre-excitation syndrome, ventricular flutter, ventricular fibrillation, ventricular escape, first degree atrio-ventricular block, secondary degree atrio-ventricular block, third degree atrio-ventricular block, intra-ventricular block, left bundle branch block, complete right bundle branch block, conduction block in left forearm, left ventricular hypertrophy, right ventricular hypertrophy, left atrial hypertrophy and right atrial hypertrophy.

In some alternative embodiments, the M preset paroxysmal heart diseases may be M paroxysmal heart diseases selected from a preset paroxysmal heart disease set including: paroxysmal sinus tachycardia, paroxysmal sinus bradycardia, paroxysmal premature atrial contraction, paroxysmal premature junctional contraction, paroxysmal premature ventricular contraction, paroxysmal supraventricular tachycardia, paroxysmal ventricular tachycardia, paroxysmal atrial flutter, paroxysmal atrial fibrillation, paroxysmal atrial escape, paroxysmal junctional escape, paroxysmal ventricular escape, paroxysmal sinus arrhythmia, paroxysmal sinus arrest and paroxysmal supraventricular premature beats.

It should be noted that implementation details and technical effects of the units of the electrocardiogram analysis apparatus provided by the present disclosure can refer to the description of other embodiments of the present disclosure, which will not be described in detail herein.

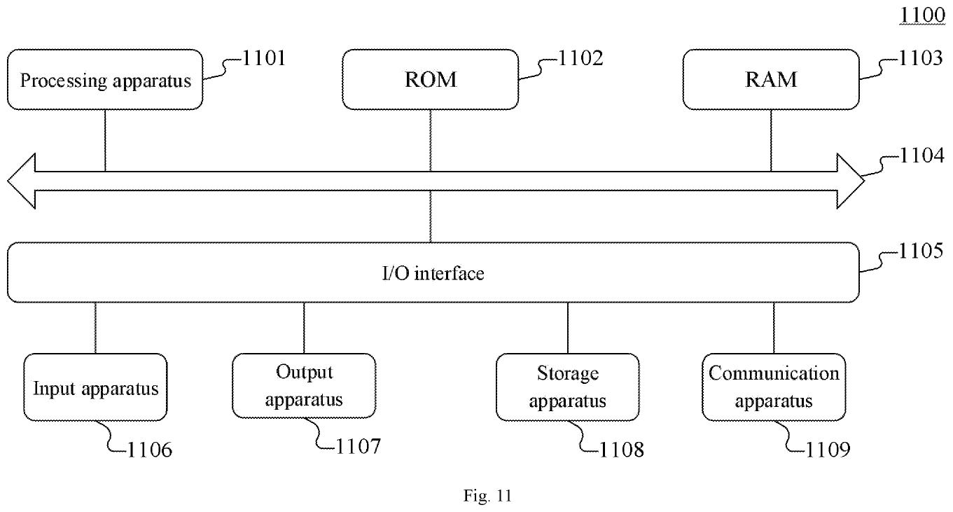

Referring to FIG. 11 below, a schematic structural diagram of a computer system 1100 suitable for implementing an electronic device in an embodiment of the present disclosure is shown. The computer system 1100 shown in FIG. 11 is only one example and should not impose any limitation on the functions and the scope of use of the embodiments of the present disclosure. As shown in FIG. 11, the computer system 1100 may include a processing apparatus (e.g. a central processor, a graphics processor, etc.) 1101 that may perform various suitable actions and processes in accordance with programs stored in a read only memory (ROM) 1102 or loaded from a storage apparatus 1108 into a random access memory (RAM) 1103. In the RAM 1103, various programs and data required for the operation of the electronic device are also stored. The processing apparatus 1101, the ROM 1102 and the RAM 1103 are connected to each other via a bus 1104. An input/output (I/O) interface 1105 is also connected to the bus 1104.

In general, the following apparatuses may be connected to the I/O interface 1105: an input apparatus 1106 including, for example, a touch screen, a touch pad, a keyboard, a mouse, a camera, a microphone, etc.; an output apparatus 1107 including, for example, a liquid crystal display (LCD), a speaker, a vibrator, etc.; a storage apparatus 1108 including, for example, a magnetic tape, a hard disk, etc.; and a communication apparatus 1109. The communication apparatus 1109 may allow the computer system 1100 to communicate wirelessly or wiredly with other devices to exchange data. Although FIG. 11 illustrates a computer system 1100 with various apparatuses, it is to be understood that not all illustrated apparatuses are required to be implemented or provided. More or fewer apparatus may alternatively be implemented or provided.

In particular, processes described above with reference to flowcharts may be implemented as computer software programs in accordance with embodiments of the present disclosure. For example, embodiments of the present disclosure include a computer program product including a computer program embodied on a computer-readable medium, the computer program including a program code for performing the methods illustrated in the flowcharts. In such embodiments, the computer program may be downloaded and installed from a network via the communication apparatus 1109, or installed from the storage apparatus 1108, or installed from the ROM 1102. The computer program, when executed by the processing apparatus 1101, performs the above-described functions defined in the method of embodiments of the present disclosure.

It should be noted that the computer-readable medium described above in the present disclosure can be either a computer-readable signal medium or a computer-readable storage medium or any combination thereof. The computer-readable storage medium can be, for example but is not limited to, an electronic, magnetic, optical, electromagnetic, infrared, or semiconductor system, apparatus, or device, or a combination thereof. More specific examples of the computer-readable storage medium may include, but are not limited to: an electrical connection having one or more wires, a portable computer diskette, a hard disk, a random access memory (RAM), a read-only memory (ROM), an erasable programmable read-only memory (EPROM or flash memory), an optical fiber, a portable compact disk read-only memory (CD-ROM), an optical storage device, a magnetic storage device, or any suitable combination thereof. In the present disclosure, the computer-readable storage medium may be any tangible medium that can contain or store a program that can be used by or in connection with an instruction execution system, apparatus, or device. In the present disclosure, the computer-readable signal medium may include a data signal embodied in a baseband or propagated as part of a carrier wave carrying a computer-readable program code. Such propagated data signals may take many forms, including but not limited to electromagnetic signals, optical signals, or any suitable combination thereof. The computer-readable signal medium can also be any computer-readable medium other than the computer-readable storage medium, and the computer-readable signal medium can send, propagate, or transport the program for being used by or in connection with the instruction execution system, apparatus, or device. The program code embodied on the computer-readable medium may be transmitted over any suitable medium including, but not limited to: wires, optical cables, RF (radio frequency), and the like, or any suitable combination thereof.

The computer-readable medium may be included in the electronic device; and may also be present separately and not fitted into the electronic device.

The computer-readable medium carries one or more programs which, when executed by the electronic device, cause the electronic device to perform the electrocardiogram analysis method as shown in the embodiment of FIG. 2A, FIG. 5 or FIG. 8 and its alternative embodiments.

A computer program code for carrying out operations of the present disclosure may be written in one or more programming languages, including object-oriented programming languages, such as Java, Smalltalk, and C++, and conventional procedural programming languages, such as the “C” language or similar programming languages. The program code may be executed entirely on the user's computer, partly on the user's computer, as a stand-alone software package, partly on the user's computer and partly on a remote computer, or entirely on the remote computer or server. In the case of the remote computer, the remote computer may be connected to the user's computer through any kind of network, including a LAN or a WAN, or may be connected to an external computer (e.g. through an Internet using an Internet Service Provider).

The flowcharts and block diagrams in the Figures illustrate the architecture, function, and operation of possible implementations of systems, methods, and computer program products according to various embodiments of the present disclosure. In this regard, each block in the flowchart or block diagrams may represent one module, program segment, or portion of a code, which includes one or more executable instructions for implementing the specified logical function(s). It should also be noted that in some alternative implementations, the functions noted in the blocks may also occur in a different order from those noted in the figures. For example, two blocks shown in succession may, in fact, be executed substantially in parallel, and sometimes in the reverse order, depending upon the function involved. It will also be noted that each block of the block diagrams and/or flowcharts, and combinations of blocks in the structural diagrams and/or flowcharts, can be implemented by special purpose hardware-based systems which perform the specified functions or operations, or combinations of special purpose hardware and computer instructions.

The units described in the embodiments of the present disclosure may be implemented in software or hardware. Where the name of the unit does not constitute a limitation on the unit itself in some cases, for example, the data acquisition unit may also be described as “a unit for acquiring at least one electrocardiogram data segment to be analyzed of a target user”.

The foregoing description is only illustrative of the preferred embodiments of the present disclosure and of the principles of the technology employed. It will be understood by those skilled in the art that the scope of disclosure involved in the present disclosure is not limited to the technical solutions formed by the particular combination of the technical features described above, but also covers other technical solutions formed by any combinations of the technical features described above or their equivalents without departing from the conception of the present disclosure. For example, a technical solution formed by the condition that the above-mentioned features and the technical features having similar functions disclosed in the present disclosure (but not limited to) are replaced with each other.

What is claimed is:

1. An electrocardiogram analysis method, including:
acquiring at least one electrocardiogram data segment to be analyzed of a target user;
inputting each electrocardiogram data segment to be analyzed into a pre-trained first electrocardiogram analysis model to obtain a heart disease suffering probability vector corresponding to the electrocardiogram data segment to be analyzed, wherein the heart disease suffering probability vector is used for characterizing a probability of suffering from each of K preset heart diseases, the first electrocardiogram analysis model is used for characterizing a correspondence between the electrocardiogram data segment and the heart disease suffering probability vector, and K is a positive integer; and
generating heart disease diagnosis result information of the target user on the basis of the heart disease suffering probability vector corresponding to each electrocardiogram data segment to be analyzed.

2. The method according to claim 1, wherein the first electrocardiogram analysis model is pre-trained with the following first training steps:
acquiring a first training data set, wherein first training data includes a sample electrocardiogram data segment and a corresponding labeled heart disease suffering probability vector, and the labeled heart disease suffering probability vector in the first training data is used for indicating a probability of a person, whom the sample electrocardiogram data segment in the first training data corresponds to and on whom collection is performed, suffering from each preset heart disease;
training an initial first electrocardiogram analysis model on the basis of the first training data set; and
determining the initial first electrocardiogram analysis model trained as the pre-trained first electrocardiogram analysis model.

3. The method according to claim 1, wherein the generating heart disease diagnosis result information of the target user on the basis of the heart disease suffering probability vector corresponding to each electrocardiogram data segment to be analyzed includes:
for each preset heart disease, performing the following first diagnosis result information generating operations: determining a heart disease suffering probability of the target user suffering from the preset heart disease according to a component corresponding to the preset heart disease in the heart disease suffering probability vector corresponding to each electrocardiogram data segment to be analyzed; determining heart disease diagnosis result information corresponding to the heart disease suffering probability of the target user suffering from the preset heart disease according to a correspondence between a disease suffering probability range and heart disease diagnosis result information corresponding to the preset heart disease; and generating heart disease diagnosis result information of the target user suffering from the preset heart disease using the determined heart disease diagnosis result information;
wherein the determining a heart disease suffering probability of the target user suffering from the preset heart disease according to a component corresponding to the preset heart disease in the heart disease suffering probability vector corresponding to each electrocardiogram data segment to be analyzed includes:
determining a mean value of components corresponding to the preset heart disease in the heart disease suffering probability vector corresponding to each electrocardiogram data segment to be analyzed as the heart disease suffering probability of the target user suffering from the preset heart disease; or
ranking components corresponding to the preset heart disease in the heart disease suffering probability vector corresponding to each electrocardiogram data segment to be analyzed in an order from low to high, and determining a component ranked in a preset quantile as the heart disease suffering probability of the target user suffering from the preset heart disease; or ranking components corresponding to the preset heart disease in the heart disease suffering probability vector corresponding to each electrocardiogram data segment to be analyzed in an order from low to high;
in response to determining that current application scenario is a less false positive scenario, determining a minimum value of the components corresponding to the preset heart disease in the heart disease suffering probability vector corresponding to each electrocardiogram data segment to be analyzed or a component ranked in a preset lower probability quantile as the heart disease suffering probability of the target user suffering from the preset heart disease; and
in response to determining that the current application scenario is a less false negative scenario, determining a maximum value of the components corresponding to the preset heart disease in the heart disease suffering probability vector corresponding to each electrocardiogram data segment to be analyzed or a component ranked in a preset higher probability quantile as the heart disease suffering probability of the target user suffering from the preset heart disease.

4. (canceled)

5. (canceled)

6. The method according to claim 3, wherein the correspondence between a disease suffering probability range and heart disease diagnosis result information corresponding to the preset heart disease includes at least one of:
a first correspondence for characterizing that a first disease suffering probability range corresponds to first diagnosis result information indicating that the preset heart disease is not diagnosed, wherein the first disease suffering probability range is less than a disease suffering probability threshold corresponding to the preset heart disease; and
a second correspondence for characterizing that a second disease suffering probability range corresponds to second diagnosis result information indicating that the preset heart disease is diagnosed, wherein the second disease suffering probability range is greater than or equal to a disease suffering probability threshold corresponding to the preset heart disease.

7. The method according to claim 6, wherein the disease suffering probability threshold corresponding to each preset heart disease is obtained by the following disease suffering probability threshold determination steps:
acquiring a test data set, wherein test data includes a sample electrocardiogram data segment and a labeled heart disease suffering probability vector, and the labeled heart disease suffering probability vector in the test data is used for indicating a probability of a person, whom the sample electrocardiogram data segment in the test data corresponds to and on whom collection is performed, suffering from each preset heart disease;
inputting sample electrocardiogram data segments in each of the test data into the first electrocardiogram analysis model to obtain a heart disease suffering probability vector test result corresponding to the test data; and
for each preset heart disease, performing the following disease suffering probability threshold determination operations: acquiring a set of candidate disease suffering probability thresholds corresponding to the preset heart disease; for each candidate disease suffering probability threshold acquired, performing the following statistical operations: according to whether a vector component corresponding to the preset heart disease in the heart disease suffering probability vector test result corresponding to each of the test data is greater than the candidate disease suffering probability threshold, and whether a vector component corresponding to the preset heart disease in a labeled heart disease suffering probability vector in the corresponding test data is greater than the candidate disease suffering probability threshold, counting a sensitivity and specificity corresponding to the preset heart disease and the candidate disease suffering probability threshold; in response to determining that the current application scenario is a less false negative scenario, ranking the candidate disease suffering probability thresholds in the set of candidate disease suffering probability thresholds corresponding to the preset heart disease in an order of the corresponding sensitivity from high to low; determining a candidate disease suffering probability threshold ranked at a preset higher sensitivity ranking position in the set of candidate disease suffering probability thresholds corresponding to the preset heart disease as a heart disease suffering probability threshold corresponding to the preset heart disease; in response to determining that the current application scenario is a less false positive scenario, ranking the candidate disease suffering probability thresholds in the set of candidate disease suffering probability thresholds corresponding to the preset heart disease in an order of the corresponding specificity from high to low; and determining a candidate disease suffering probability threshold ranked at a preset higher specificity ranking position in the set of candidate disease suffering probability thresholds corresponding to the preset heart disease as a heart disease suffering probability threshold corresponding to the preset heart disease.

8. The method according to claim 1, wherein the generating heart disease diagnosis result information of the target user on the basis of the heart disease suffering probability vector corresponding to each electrocardiogram data segment to be analyzed includes:
for each preset heart disease, performing the following second diagnosis result information generating operations: acquiring a set of disease suffering probability ranges corresponding to the preset heart disease; for each acquired disease suffering probability range, determining a proportion of data segments corresponding to the disease suffering probability range, wherein the proportion of data segments corresponding to the disease suffering probability range is a proportion of the number of components of the heart disease suffering probability vector belonging to the disease suffering probability range in the components corresponding to the preset heart disease in the heart disease suffering probability vector corresponding to each electrocardiogram data segment to be analyzed divided by the number of the electrocardiogram data segments to be analyzed; according to a correspondence between a disease suffering probability range and heart disease diagnosis result information corresponding to the preset heart disease, determining heart disease diagnosis result information corresponding to the disease suffering probability range with the largest proportion of the corresponding data segments; and
generating heart disease diagnosis result information of the target user suffering from the preset heart disease using the determined heart disease diagnosis result information.

9. The method according to claim 1, wherein the generating heart disease diagnosis result information of the target user on the basis of the heart disease suffering probability vector corresponding to each electrocardiogram data segment to be analyzed includes:
for each preset heart disease, in response to determining that a proportion of diagnosis electrocardiogram data segments corresponding to the preset heart disease is not less than a diagnosis proportion threshold corresponding to the preset heart disease, labeling the preset heart disease as a diagnosed heart disease, wherein the proportion of diagnosis electrocardiogram data segments corresponding to the preset heart disease is a proportion of the number of diagnosis electrocardiogram data segments corresponding to the preset heart disease divided by the total number of the electrocardiogram data segments to be analyzed, and the number of diagnosis electrocardiogram data segments corresponding to the preset heart disease is the number of components corresponding to the preset heart disease in a heart disease suffering probability vector corresponding to each electrocardiogram data segment to be analyzed, which are greater than a disease suffering probability threshold corresponding to the preset heart disease; and
generating heart disease diagnosis result information indicating that the target user is diagnosed with a diagnosed heart disease among the preset heart diseases, which is labeled as a diagnosed heart disease.

10. The method according to claim 1, wherein the method further includes:
for each of M preset paroxysmal heart diseases, in response to the target user's heart disease diagnosis result information indicating that a probability of the target user suffering from a heart disease corresponding to the preset paroxysmal heart disease falls within a preset lower disease suffering probability range, performing the following first paroxysmal heart disease prediction operations for the preset paroxysmal heart disease: calculating a probability vector distance between a heart disease suffering probability vector of the target user and a reference paroxysmal heart disease suffering probability vector corresponding to the preset paroxysmal heart disease; and generating paroxysmal heart disease diagnosis result information for indicating that the target user suffers from the preset paroxysmal disease in response to determining that the probability vector distance is less than a probability vector distance threshold corresponding to the preset paroxysmal heart disease, wherein M is a positive integer less than or equal to K, and heart diseases corresponding to the M preset paroxysmal heart diseases belong to the K preset heart diseases.

11. The method according to claim 10, wherein the first paroxysmal heart disease prediction operations further include:
in response to determining that the probability vector distance is not less than the probability vector distance threshold corresponding to the preset paroxysmal heart disease, generating paroxysmal heart disease diagnosis result information indicating that the target user does not suffer from the preset paroxysmal disease.

12. The method according to claim 10, wherein the reference paroxysmal heart disease suffering probability vector corresponding to each preset paroxysmal heart disease is obtained by performing the following probability vector generation steps for each of the preset paroxysmal heart diseases:
acquiring a set of unacknowledged condition electrocardiogram data segments corresponding to the preset paroxysmal heart disease, wherein each of the unacknowledged condition electrocardiogram data segments is an electrocardiogram data segment obtained after segmenting unacknowledged condition electrocardiogram data, and the unacknowledged condition electrocardiogram data is electrocardiogram data of electrocardiogram examination on a subject diagnosed with a heart disease corresponding to the paroxysmal heart disease, which is labeled as the subject corresponding to the unacknowledged condition electrocardiogram data does not suffer from the heart disease corresponding to the paroxysmal heart disease;
inputting each unacknowledged condition electrocardiogram data segment into the first electrocardiogram analysis model to obtain a corresponding heart disease suffering probability vector;
for each of the unacknowledged condition electrocardiogram data segments, determining a probability vector average distance corresponding to the unacknowledged condition electrocardiogram data segment, wherein the probability vector average distance corresponding to the unacknowledged condition electrocardiogram data segment is an average distance between a heart disease suffering probability vector corresponding to the unacknowledged condition electrocardiogram data segment and heart disease suffering probability vectors corresponding to other unacknowledged condition electrocardiogram data segments in the unacknowledged condition electrocardiogram data segment set except for the unacknowledged condition electrocardiogram data segment;
determining a central unacknowledged condition electrocardiogram data segment in each of the unacknowledged condition electrocardiogram data segments on the basis of a probability vector average distance corresponding to each unacknowledged condition electrocardiogram data segment; and
determining a heart disease suffering probability vector corresponding to the central unacknowledged condition electrocardiogram data segment as a reference paroxysmal heart disease suffering probability vector corresponding to the paroxysmal heart disease.

13. The method according to claim 12, wherein the probability vector distance threshold corresponding to each of the M preset paroxysmal heart diseases is obtained by:
ranking the unacknowledged condition electrocardiogram data segments in an order of the corresponding probability vector average distance from high to low;
determining an unacknowledged condition electrocardiogram data segment, ranked at a preset boundary probability vector average distance ranking position, of the unacknowledged condition electrocardiogram data segments as a boundary unacknowledged condition electrocardiogram data segment; and
for each of the M preset paroxysmal heart diseases, determining a component, which is corresponding to the heart disease corresponding to the paroxysmal heart disease, of a heart disease suffering probability vector corresponding to the boundary unacknowledged condition electrocardiogram data segment as a probability vector distance threshold corresponding to the paroxysmal heart disease.

14. The method according to claim 1, wherein the method further includes:
for each of M preset paroxysmal heart diseases, in response to the target user's heart disease diagnosis result information indicating that a probability of the target user suffering from a heart disease corresponding to the preset paroxysmal heart disease falls within a preset lower disease suffering probability range, performing the following second paroxysmal heart disease prediction operations: inputting each electrocardiogram data segment to be analyzed into a pre-trained second electrocardiogram analysis model corresponding to the preset paroxysmal heart disease to obtain a paroxysmal heart disease prediction result corresponding to the preset paroxysmal heart disease and the electrocardiogram data segment to be analyzed for characterizing whether the preset paroxysmal heart disease exists, wherein the second electrocardiogram analysis model corresponding to the preset paroxysmal heart disease is used for characterizing a correspondence between the electrocardiogram data segment and the paroxysmal heart disease prediction result; and generating a paroxysmal heart disease prediction result of the target user for the preset paroxysmal heart disease on the basis of the paroxysmal heart disease prediction result corresponding to the preset paroxysmal heart disease and each electrocardiogram data segment to be analyzed, wherein M is a positive integer less than or equal to K, and heart diseases corresponding to the M preset paroxysmal heart diseases belong to the K preset heart diseases.

15. The method according to claim 14, wherein the generating a paroxysmal heart disease prediction result of the target user for the preset paroxysmal heart disease on the basis of the paroxysmal heart disease prediction result corresponding to the preset paroxysmal heart disease and each electrocardiogram data segment to be analyzed includes:
determining whether a paroxysmal heart disease prediction result indicating suffering from the preset paroxysmal heart disease exists in the paroxysmal heart disease prediction results corresponding to the preset paroxysmal heart disease and each electrocardiogram data segment to be analyzed; and in response to determining presence, generating a paroxysmal heart disease prediction result indicating that the target user suffers from the preset paroxysmal heart disease.

16. The method according to claim 15, wherein the generating a paroxysmal heart disease prediction result of the target user for the preset paroxysmal heart disease on the basis of the paroxysmal heart disease prediction result corresponding to the preset paroxysmal heart disease and each electrocardiogram data segment to be analyzed further includes:
in response to determining absence, generating a paroxysmal heart disease prediction result indicating that the target user does not suffer from the preset paroxysmal heart disease.

17. The method according to claim 14, wherein the generating a paroxysmal heart disease prediction result of the target user for the preset paroxysmal heart disease on the basis of the paroxysmal heart disease prediction result corresponding to the preset paroxysmal heart disease and each electrocardiogram data segment to be analyzed includes:
generating a paroxysmal heart disease prediction result indicating that the target user suffers from the preset paroxysmal heart disease in response to determining that a proportion of diagnosis prediction results corresponding to the preset paroxysmal heart disease is greater than a diagnosis prediction result proportion threshold corresponding to the preset paroxysmal heart disease, wherein the proportion of diagnosis prediction results corresponding to the preset paroxysmal heart disease is a proportion of the number of diagnosis prediction results corresponding to the preset paroxysmal heart disease divided by the total number of the electrocardiogram data segments to be analyzed, and the number of the diagnosis prediction results corresponding to the preset paroxysmal heart disease is the number of paroxysmal heart disease prediction results indicating suffering from the preset paroxysmal heart disease in the paroxysmal heart disease prediction results corresponding to the preset paroxysmal heart disease and the electrocardiogram data segments to be analyzed.

18. The method according to claim 17, wherein the generating a paroxysmal heart disease prediction result of the target user for the preset paroxysmal heart disease on the basis of the paroxysmal heart disease prediction result corresponding to the preset paroxysmal heart disease and each electrocardiogram data segment to be analyzed further includes:
generating a paroxysmal heart disease prediction result indicating that the target user does not suffer from the preset paroxysmal heart disease in response to determining that the proportion of diagnosis prediction results corresponding to the preset paroxysmal heart disease is not greater than the diagnosis prediction result proportion threshold corresponding to the preset paroxysmal heart disease.

19. The method according to claim 1, wherein the acquiring at least one electrocardiogram data segment to be analyzed of a target user includes:
acquiring electrocardiogram data to be analyzed of a target user; and
segmenting the electrocardiogram data to be analyzed to obtain at least one electrocardiogram data segment to be analyzed.

20. The method according to claim 19, wherein before the segmenting the electrocardiogram data to be analyzed to obtain at least one electrocardiogram data segment to be analyzed, the method further includes:
resampling the electrocardiogram data to be analyzed so that a sampling frequency of the electrocardiogram data to be analyzed is a preset sampling frequency.

21. (canceled)

22. The method according to claim 1, wherein the K preset heart diseases are K heart diseases selected from a preset heart disease set including: sinus tachycardia, sinus bradycardia, premature atrial contraction, premature junctional contraction, premature ventricular contraction, supraventricular tachycardia, ventricular tachycardia, atrial flutter, atrial fibrillation, atrial escape, junctional escape, ventricular escape, right bundle branch block, sinus arrhythmia, sinus arrest, supraventricular premature beats, paired supraventricular premature beats, bigeminy coupled rhythm of supraventricular premature beats, trigeminy of supraventricular premature beats, ventricular premature beats, paired ventricular premature beats, bigeminy coupled rhythm of ventricular premature beats, trigeminy of ventricular premature beats, supraventricular escape beats, pre-excitation syndrome, ventricular flutter, ventricular fibrillation, ventricular escape, first degree atrio-ventricular block, secondary degree atrio-ventricular block, third degree atrio-ventricular block, intra-ventricular block, left bundle branch block, complete right bundle branch block, conduction block in left forearm, left ventricular hypertrophy, right ventricular hypertrophy, left atrial hypertrophy and right atrial hypertrophy.

23. The method according to claim 18, wherein the M preset paroxysmal heart diseases are M paroxysmal heart diseases selected from a preset paroxysmal heart disease set including: paroxysmal sinus tachycardia, paroxysmal sinus bradycardia, paroxysmal premature atrial contraction, paroxysmal premature junctional contraction, paroxysmal premature ventricular contraction, paroxysmal supraventricular tachycardia, paroxysmal ventricular tachycardia, paroxysmal atrial flutter, paroxysmal atrial fibrillation, paroxysmal atrial escape, paroxysmal junctional escape, paroxysmal ventricular escape, paroxysmal sinus arrhythmia, paroxysmal sinus arrest and paroxysmal supraventricular premature beats.

24. An electrocardiogram analysis apparatus, including:
a data acquisition unit, configured to acquire at least one electrocardiogram data segment to be analyzed of a target user;
a data analysis unit, configured to input each electrocardiogram data segment to be analyzed into a pre-trained first electrocardiogram analysis model to obtain a heart disease suffering probability vector corresponding to the electrocardiogram data segment to be analyzed, wherein the heart disease suffering probability vector is used for characterizing a probability of suffering from each of K preset heart diseases, the first electrocardiogram analysis model is used for characterizing a correspondence between the electrocardiogram data segment and the heart disease suffering probability vector, and K is a positive integer; and
a heart disease diagnosis result generation unit, configured to generate heart disease diagnosis result information of the target user on the basis of the heart disease suffering probability vector corresponding to each electrocardiogram data segment to be analyzed.

25. An electronic device, including:
one or more processors; and
a storage apparatus having one or more programs stored thereon, wherein the one or more programs, when executed by the one or more processors, cause the one or more processors to implement the method according to claim 1.

26. A computer-readable storage medium, having a computer program stored thereon, wherein the computer program, when executed by one or more processors, implements the method according to claim 1.
